(19) United States
(12) Patent Application Publication
(10) Pub. No.: US 2016/0287638 A1
(43) Pub. Date: Oct. 6, 2016

(54) ISCHEMIC TOLERANT CELLS AND CELLULAR FACTORS IN THE TREATMENT OF PERIPHERAL ARTERY DISEASE

(71) Applicant: Stemedica Cell Technologies, Inc., San Diego, CA (US)

(72) Inventors: Stephen E. Epstein, Rockville, MD (US); Alex Kharazi, San Diego, CA (US); Nikolai Tankovich, San Diego, CA (US); Sergey Sikora, San Diego, CA (US)

(73) Assignee: Stemedica Cell Technologies, Inc., San Diego, CA (US)

(21) Appl. No.: 14/677,399

(22) Filed: Apr. 2, 2015

(51) Int. Cl. A61K 35/28; A61K 9/00

(52) U.S. Cl. CPC A61K 35/28; A61K 9/0019

(57) ABSTRACT

A method for treatment of peripheral arterial disease conditions comprising systemically administering an effective amount of therapeutic stem cells. Included within the scope of the invention is intravenous administration of a therapeutic amount of ischemic tolerant mesenchymal stem cells in the treatment of peripheral arterial disease.

### FIELD OF THE INVENTION

The invention relates to the use of stem cells in the treatment of limb ischemia. More particularly, the invention relates to a method of using ischemic tolerant cells, and cellular factors derived therefrom, in the treatment of peripheral artery disease and related ischemic limb conditions.

### BACKGROUND

Peripheral arterial disease is associated with significant morbidity and mortality. There is no pharmacological therapy available, but several reports have suggested that mesenchymal stem cells (MSCs) may be a useful therapeutic option. Peripheral artery disease (PAD) is estimated to affect more than 27 million people in North American and Europe.

Stem cell therapies hold promise as novel therapeutics to promote vasculogenesis and improve tissue perfusion in these patients. Among those in this field, the types of stem cells and their routes of administration for treatment of PAD is an unsettled matter. The two major routes of administration have been intramuscular (IM) and intra-arterial (IA). To date, the majority of cell therapy trials for PAD have relied upon IM delivery. IM MSC transplantation has been shown to induce neovascularization in a rat model of hindlimb ischemia. There is a need to assess the therapeutic potency of intravenous MSC administration in a mouse model of hindlimb ischemia, to evaluate the angiogenic potential of MSC in subjects with PAD.

### SUMMARY OF THE INVENTION

An objective of the invention is to provide a method for the treatment of peripheral artery disease in a patient comprising intravenously administering a therapeutically effective amount of ischemic tolerant mesenchymal stem cells. Certain embodiments of the invention involve a step of intramuscular injection in addition to intravenous injection.

It is an object of the present invention to provide a therapeutic method for treating peripheral arterial disease.

### FIGURES

FIG. 1 is a mouse hindlimb ischemia model in which in vitro imaging system (IVIS) tracked the distribution of labeled itMSCs injected intravenously.

FIG. 2 IV MSC injection of mouse24 hours after MI

FIG. 3 IV injection of MSC in mouse without MI

FIG. 4 comparison of percent increase in intensity of gamma signal

FIG. 5 compares, the total gamma signal present to the percent of the LV infarcted.

FIG. 6 shows a frontal view of radioactivity in the heart and GI tract (GI activity reflecting degraded indium-111 oxine).

FIG. 7 ex vivo imaging of heart 24 hr post human MSC injection

FIG. 8 flow cytometry of human MSC labeled with Q-dos and injected iv into mice with MI

FIG. 9 distribution of labeled cells

FIG. 10 diffuse distribution of cells to multiple tissues occurs even with intramyocardial and intracoronary injection of cells

FIG. 11 MSCs present in ischemic myocardium 7 days after injection compared to 24 hours after injection.

FIG. 12 Increased engraftment of MSCs in ischemic and non-ischemic tissue following repeated iv injections.

FIG. 13 flow cytometry analysis of myocardium performed seven days post MSC injection.

FIG. 14 prior art of coronary arteriole plug occurring consequent to intracoronary injection of MSC in a porcine model of AMI

FIG. 15 Prior art study of chronic MI in porcine model 4 weeks post-acute AMI; coronary arteriole plug occurring consequent to intracoronary injection of MSC in a porcine model of AMI

FIG. 16 Prior art study of chronic MI in porcine model 4 weeks post-acute AMI

FIG. 17 IV delivery of human MSCs grown under chronic hypoxic conditions into mice with an AMI that occurred 2 months earlier

FIG. 18 silk ligature in place and tied on left coronary artery of rat

FIG. 19 normal EKG of rat

FIG. 20 Displacement of the ST segment

FIG. 21 displacement of the ST segment in rat EKG showing the acute stage of myocardial infarction when the high ST segment merges with the increased positive T wave forming a monophasic curve. These are EKGs of rats in the acute stage of myocardial infarction.

FIG. 22 rat EKG in acute phase showing appearance of Q wave

FIG. 23 rat EKG shows formation of expansive areas of necrosis in the heart muscle. In some cases, the QRS complex was missing and formed a QS complex

FIG. 24 rat EKG shows QS complex

FIG. 25 Schematic diagram of heart showing position cuts made at 4, 6, and 8 mm from the apex

FIG. 26 shows change in the rat weight during the course of the experiment.

FIG. 27 histological preparation of rat myocardium of the control group

FIG. 28 histological section of a healthy rat myocardial longitudinal section of muscle fibers.

FIG. 29 Morphology of the rat myocardium of the left and right ventricle following experimental myocardial infarction

FIG. 30 Morphology of the rat myocardium of the left and right ventricle following experimental myocardial infarction

FIG. 31 Large and small coronary arteries and veins in a state of severe congestion

FIG. 32 Morphology of the myocardium of the left and right ventricle after myocardial infarct

FIG. 33 myocardial infarcted tissue in early stage of scarring

FIG. 34 rat left ventricle with discernible connective scar tissue

FIG. 35 rat left ventricle myocardium

FIG. 36 rat left ventricle myocardium

FIG. 37 cicatrical tissue

FIG. 38 Morphological pattern of the left and right rat ventricle of the myocardium after experimental myocardial infarction and treated with cellular factors

FIG. 39 Morphological pattern of the left and right rat ventricle of the myocardium after experimental myocardial infarction and treated with cellular factors

FIG. 40 Morphological pattern of the left and right rat ventricle of the myocardium after experimental myocardial infarction and treated with cellular factors

FIG. 41 Morphological pattern of the left and right rat ventricle of the myocardium after experimental myocardial infarction and treated with cellular factors

FIG. 42 transverse sections of the heart clearly show the difference in infarct size between the SC group IM group, which exhibit a decrease in the zone of the affected myocardium.

FIG. 43 length of the infarction as measure of circumference of wall of left ventricle deformed to postinfaraction cardiosclerosis

FIG. 44 magnitude of heart attack

FIG. 45 The amount of scar tissue area was significantly lower in Group SC and CF compared with the IM group by 36.7% and 20.8% respectively

FIG. 46 increase in the volume density of the functioning myocardial infarction and scar tissue compared to the group without treatment by 9.6%

FIG. 47 The volume density of the ventricular cavities and leukocyte infiltration

FIG. 48 Elisa data shows the level of C-reactive protein had a tendency to decrease in CF groups compared with the control group MI and the SC group. However, no significant differences were found.

FIG. 49 Elisa data shows the level of C-reactive protein had a tendency to decrease in CF groups compared with the control group MI and the SC group. However, no significant differences were found.

### DESCRIPTION

### Definitions

As used herein, the term “stem cell” refers to an undifferentiated cell which has the ability to both self-renew (through mitotic cell division) and undergo differentiation to form a more specialized cell. Stem cells have varying degrees of potency. A precursor cell is but one example of a stem cell.

As used herein, the term “mesenchymal cell” refers to mesodermal germ lineage cells which may or may not be differentiated. The mesenchymal cells of the invention include cells at all stages of differentiation beginning with multipotent mesenchymal stem cells, down to fully differentiated terminal cells. Mesenchymal stem cells can be sourced from embryonic stem cells, and iPS cells.

Induced pluripotent stem cells (also known as iPS cells or iPSCs) are a type of pluripotent stem cell that can be generated directly from adult cells. Induced pluripotent stem cells (iPSCs) are adult cells that have been genetically reprogrammed to an embryonic stem cell-like state by being forced to express genes and factors important for maintaining the defining properties of embryonic stem cells. iPSCs demonstrate important characteristics of pluripotent stem cells, including expressing stem cell markers, forming tumors containing cells from all three germ layers, and being able to contribute to many different tissues when injected into mouse embryos at a very early stage in development. Human iPSCs also express stem cell markers and are capable of generating cells characteristic of all three germ layers, including mesodermal stem cells, i.e. mesenchymal stem cells. An embodiment of the method of the invention administers compositions comprising mesenchymal stem cells which are genetically reprogrammed de novo for variant expression of growth factors and cytokines.

It should be understood that the stem cell composition administered by the claimed method may comprise more than one line of mesenchymal stem cells. The stem cell composition may comprise, with respect to the patient receiving the cells, mesenchymal stem cells which are autologous or allogenic or a combination thereof.

As used herein, the term “ischemic tolerant” may be used to describe a cell, cell culture or tissue which has been exposed to atmospheric conditions having an oxygen concentration that is less than ambient air. Such exposure may include, but is in no way limited to, priming cells and/or growing cells under low oxygen conditions.

As used herein, the term “patient,” or “subject,” refers to animals, including mammals, preferably humans, who are treated with the pharmaceutical compositions or accordance with the methods described herein

As used herein, the term “pharmaceutically acceptable carrier” (or medium), which may be used interchangeably with the term “biologically compatible carrier” (or medium), refers to reagents, cells, compounds, materials, compositions, and/or dosage forms that are not only compatible with the cells and other agents to be administered therapeutically, but also are, within the scope of sound medical judgment, suitable for use in contact with the tissues of human beings and animals without excessive toxicity, irritation, allergic response, or other complication commensurate with a reasonable benefit/risk ratio.

As used herein, the term a “therapeutically effective amount,” or “effective amount,” refers to the amount of a composition that produces a therapeutic effect or improvement in a targeted disorder.

As used herein, the term “stem cell composition” refers to a composition containing whole stem cells, stem cell factors thereof or stem cell factors secreted by a separate line of stem cells, microvesicles, or a combination thereof.

In certain embodiments, the method administers an acellular composition comprising stem cell factors derived from mesenchymal stem cells which are with respect to the subject receiving treatment, allogenic, autologous or a mixture thereof.

Stem cell factors are secreted by a particular type of cell or tissue, and include proteins that are secreted by a cell or tissue at any given time under certain conditions. These secreted elements, i.e. stem cell factors, are referred to as secretome. Stem cell factors released by cells into conditioned media in vitro have been studied to better understand pathological conditions and mechanisms in vivo (Investigating the Secretome, Circulation: Cardiovascular Genetics. 2012; 5:8-18). Stem cell factors are obtained from conditioned media derived from various stem cells, e.g. itMSCs. Methods for obtaining the secretome of bone marrow mesenchymal stem cells-conditioned media are well known (Ribeiro, C. A., Salgado, A. J. et al. J. Tissue Eng Regen Med 2011 DOI: 10.1002/term.365; Ranganath, S. H. et al. Cell Stem Cell 2912 10:244-258).

As used herein, the term “treating,” or “treat,” refers to producing a therapeutic effect in a targeted condition through the administration of an effective amount of a composition. Such therapeutic effects include preventing a pathologic condition from occurring, inhibiting the pathologic condition or arresting its development, relieving (e.g. curing or reversing) a pathologic condition, or alleviating the symptoms associated with a pathological condition.

As used herein, the term “clone,” or “clonal cell,” refers to a single cell which is expanded to produce an isolated population of phenotypically similar cells (i.e. a “clonal cell population”).

As used herein, the term “cell line” refers to one or more generations of cells which are derived from a clonal cell.

The terms “administration” and “administering” as used herein refer to the delivery of therapeutic composition by an administration route including, but not limited to, intravenous, intra-arterial, intramuscular, intraperitoneal, subcutaneous, intramuscular, topically, or combinations thereof. Certain embodiments of the method are limited to administration only by the intravenous route; or by only an intramuscular route; or by intravenous and intramuscular routes.

Peripheral Arterial Disease

As used herein, peripheral artery disease (PAD) is a narrowing of arteries that occurs most often in the legs. Plaque, a substance composed of cholesterol, calcium and fibrous tissue, builds up in the arteries and hardens over time, reducing blood flow. Symptoms range from pain to difficulty fighting infection and in severe cases, tissue death. Critical limb ischemia (CLI) is the most severe form of atherosclerotic PAD.

In general, PAD is set of peripheral vascular disease (PVDs), circulation disorders that affect blood vessels outside of the heart and brain. PVD typically strikes the veins and arteries that supply the arms, legs, and organs located below the stomach. These are the blood vessels that are distant from the heart (peripheral vessels). In PVD, blood vessels are narrowed. Narrowing is usually caused by arteriosclerosis. Arteriosclerosis is a condition where plaque builds up inside a vessel. It is also called “hardening of the arteries.” Plaque decreases the amount of blood and oxygen supplied to the arms and legs. As plaque growth progresses, clots may develop. This further restricts the affected vessel. Eventually, arteries can become obstructed.

PVD that develops only in the arteries is called peripheral arterial disease (PAD). This is the most common form of PVD. PVD and PAD are often used to mean the same condition. PVD may also be referred to as: arteriosclerosis obliterans, arterial insufficiency of the legs, or claudication. PAD involves changes in blood vessel structure, causing inflammation, tissue damage, and blockages.

Symptoms of PVD

Generally, the first symptom is discomfort in the legs and feet. One may experience painful cramping, achiness, fatigue, burning, symptoms typically experienced when one walk. One may first notice them when walking quicker, with more exertion, or for long distances. The pain will intensify with activity and subside when one rests (intermittent claudication).

Intermittent claudication occurs because the muscles need more blood flow during activity. In PVD, the vessels are narrowed with plaque. They can only supply a limited amount of blood. This causes more problems during activity than at rest. Lack of blood causes pain and discomfort.

As PVD progresses, symptoms will occur more frequently, and require less exertion to bring them on. Eventually, one will experience leg pain and fatigue even at rest. Additional symptoms may occur as a result of reduced blood supply. With PVD, one may have: gangrene wounds or ulcers on the legs and feet that won't heal, leg cramps and pain when lying in bed, severe burning pain in the toes

Complications of PVD can include blood clots that obstruct small arteries, limb amputation due to tissue death in the limb, pain when the legs are elevated, severe pain that restricts mobility, wounds that don't heal.

An object of the invention is to provide a therapeutic method for treating peripheral arterial disease which relieves or ameliorates symptoms and complications of PVD.

### DETAILED DESCRIPTION

The invention relates to the intravenous administration of a composition of cells in the treatment of peripheral artery disease. In a preferred embodiment, the invention administers intravenously mesenchymal stem cells. In still a more preferred embodiment, the invention administers intravenously ischemic tolerant stem cells. Another embodiment of the present method administers mesenchymal stem cells intramuscularly in addition to intravenous administration.

Stem cells for use with the invention include mesenchymal stem cells (MSC). Such MSC may be obtained from prenatal sources, postnatal sources, and combinations thereof. Tissues for deriving a suitable MSC include, but are not limited to, bone marrow, blood (peripheral blood), dermis (e.g. dermal papillae), periosteum, synovium, peripheral blood, skin, hair root, muscle, uterine endometrium, adipose, placenta, menstrual discharge, chorionic villus, amniotic fluid and umbilical cord blood. Mesenchymal stem cells may be derived from these sources individually, or the sources may be combined (before or after enrichment) to produce a mixed population of mesenchymal stem cells from different tissue sources.

Mesenchymal stem cell compositions for use with the invention may comprise purified or non-purified mesenchymal stem cells. Mesenchymal stem cells for use with the invention include, but are in no way limited to, those described in the following references, the disclosures of which are incorporated herein by reference: U.S. Pat. No. 5,215,927; U.S. Pat. No. 5,225,353; U.S. Pat. No. 5,262,334; U.S. Pat. No. 5,240,856; U.S. Pat. No. 5,486,359; U.S. Pat. No. 5,759,793; U.S. Pat. No. 5,827,735; U.S. Pat. No. 5,811,094; U.S. Pat. No. 5,736,396; U.S. Pat. No. 5,837,539; U.S. Pat. No. 5,837,670; U.S. Pat. No. 5,827,740; U.S. Pat. No. 6,087,113; U.S. Pat. No. 6,387,367; U.S. Pat. No. 7,060,494; U.S. Pat. No. 8,790,638; Jaiswal, N., et al., J. Cell Biochem. (1997) 64(2): 295 312; Cassiede P., et al., J. Bone Miner. Res. (1996) 11(9): 1264 1273; Johnstone, B., et al., (1998) 238(1): 265 272; Yoo, et al., J. Bone Joint Sure. Am. (1998) 80(12): 1745 1757; Gronthos, S., Blood (1994) 84(12): 41644173; Basch, et al., J. Immunol. Methods (1983) 56: 269; Wysocki and Sato, Proc. Natl. Acad. Sci. (USA) (1978) 75: 2844; and Makino, S., et al., J. Clin. Invest. (1999) 103(5): 697 705.

Ischemic tolerant stem cells (e.g. MSC) for use with the invention are grown (i.e. cultured) under low oxygen conditions. Without being limited to any particular theory, culturing the stem cells under low oxygen conditions increases stem cell proliferation and enhances the production of stem cell factors beneficial in the regeneration (and rejuvenation) of tissues in vivo.

The term “low oxygen,” or “low oxygen conditions,” as used herein refers reduced oxygen tension (i.e. any oxygen concentration that is less than atmospheric oxygen). Thus, the stem cells for use with the invention may be grown in an oxygen concentration that is below about 20%, preferably below about 15%, more preferably below about 5-10%, at sea level. Low oxygen conditions may be kept as close as possible to the normal physiological oxygen conditions in which a particular stem cell would be found in vivo.

In one embodiment, the low oxygen conditions comprise an ambient (e.g. incubator) oxygen condition of between about 0.25% to about 18% oxygen. In another embodiment, the ambient oxygen conditions comprise between about 0.5% to about 15% oxygen. In still another embodiment, the low ambient oxygen conditions comprise between about 1% to about 10% oxygen. In further embodiments, the low ambient oxygen conditions comprise between about 1.5% to about 6% oxygen. Of course, these are exemplary ranges of ambient oxygen conditions to be used in culture and it should be understood that those of skill in the art will be able to employ oxygen conditions falling in any of these ranges generally or oxygen conditions between any of these ranges that mimics physiological oxygen conditions for the particular cells. Thus, one of skill in the art could set the oxygen culture conditions at 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, 15%, 15.5%, 16%, 16.5%, 17%, 17.5%, 18%, 18.5%, or any other oxygen condition between any of these figures

Methods for manufacturing stem cells under low oxygen conditions as disclosed herein are available in the art, including the methods disclosed in the following publications, the disclosures of which are incorporated herein by reference. U.S. Pat. No. 6,759,242; U.S. Pat. No. 6,846,641; U.S. Pat. No. 6,610,540; U.S. Pat. No. 8,790,638; J. Cereb. Blood Flow Metab. 2008 Sep. 28(9):1530-42; Stem Cells. 2008 May 26(5):1325-36; Exp Neurol. 2008 April 210(2):656-70; Mol. Cell. Neurosci. (2007), doi:10.1016/j.mcn.2007.04.003; Experimental Neurology 170, 317-325 (2001); and Neurosignals 2006-07, 15:259-265. Although these references disclose particular procedures and reagents, any low oxygen culture condition capable of expanding stem cells according to the invention may be used.

As noted above, the invention is practiced by administering a stem cell composition to a patient suffering from a PAD condition. As used herein, the terms “administering,” “administered” and “administer” refer to any administration route by which a stem cell composition can be administered to a patient for a therapeutic effect as disclosed herein. For example, the stem cell composition may be administered intravenously, intra-arterially, intramuscularly, intraperitoneally, subcutaneously, intramuscularly, intranasally, sublingually, or by combination thereof. In a preferred embodiment, the stem cell composition is administered intravenously. Other embodiments comprise an additional step of intramuscular administration of mesenchymal stem cells to the hypoxic musculature.

Claudication is pain caused by too little blood flow to legs or arms, and is usually a symptom of peripheral artery disease, in which the arteries that supply blood to the limbs are narrowed, usually because of atherosclerosis. Atherosclerosis occurs when arteries get thick and stiff due to a buildup of fatty deposits (plaques) on artery walls.

In an exemplary, non-limiting embodiment, the invention is practiced by intravenously administering to a patient a composition of ischemic tolerant mesenchymal stem cells (MSC). MSC for use with this non-limiting embodiment may be derived from human bone marrow grown under low oxygen conditions. The MSC may be grown under low oxygen conditions starting with a primary culture of cells, or passage 2 MSC. Such culture may be maintained under low oxygen conditions for multiple passages, up to the point of harvest for administration to a patient. It is further contemplated in such embodiment that the MSC may be primed with oxygen, or exposed to normoxic oxygen conditions, prior to administration to a patient.

### Example 1

### Culture of Low Oxygen Bone Marrow MSC

MSC were derived from the bone marrow of a healthy donor. Mononuclear cells were isolated from a fresh specimen of bone marrow using Histopague and seeded into Petri dishes. The cells were expanded in DMEM/F12 medium containing FGF-2 and 10% fetal bovine serum (FBS). The cells were tested for human pathogens and further expanded up to passage 5 under 5% oxygen conditions. Low oxygen conditions were initiated with passage 2.

### Example 2

### Treatment of Mice Having PAD

A purpose of this study was to determine the efficacy of intravenous administration of itMSCs in mice with ischemic hind limbs.

Another purpose of this study in mice was to determine the optimal dosing and gradual redistribution with late homing of iv administered MSCs to ischemic tissue.

1. Determination of the toxic iv dose of MSCs and the optimal iv dose that can be administered.

Multi-dose concept for optimizing the initial dose of MSCs administered: Once the toxic dose to a single administration of MSCs was determined, we proceeded on the basis that this total dose can be given safely if aliquots of the toxic dose are injected over 1-2 sequential days.

In a series of mice, it was determined that 6 million cells given iv as a single injection is toxic. Mice exhibited symptoms of pulmonary emboli with SOB and profound debility occurring shortly after injection. However, ⅓ the toxic dose given 3× over 3 days was well-tolerated.

2. Migration of human MSCs from tissue reservoirs to ischemic tissue—gradual redistribution to ischemic tissue.

We hypothesized that there is a gradual release of MSCs that are acutely entrapped in the lungs and other tissues, leading to migration and redistribution so that human MSCs engraft into ischemic tissue over the several days following injection. The following figures demonstrate the validity of this phenomenon.

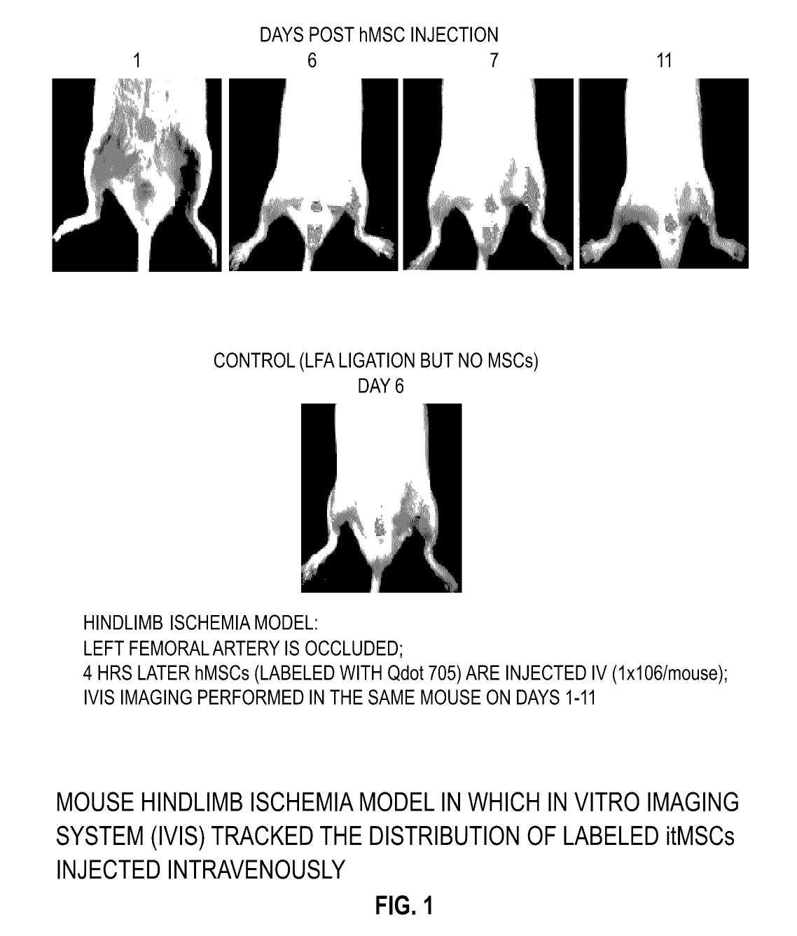

FIG. 1 represents a hindlimb ischemic model; the left femoral artery was occluded. Four hours later, human hMSCs labeled with Qdot 705 were injected iv (1×106/mouse). IVIS imaging was performed in the same mouse on days 1-11. The in vivo imaging system (IVIS) uses bioluminescent and fluorescent reporters to identify and semi-quantitate label present in vivo or ex vivo.

a) As summarized in FIG. 1, we initially employed an acute femoral artery ligation model to produce ischemia, because the resulting hindlimb ischemia occurs relatively superficially; this enabled us, using IVIS, to make serial in vivo assessments of MSCs as they redistributed over time in the same mouse.

FIG. 1 shows the finding in an iv MSC injected mouse. Day 1: No labeled cells are present in the ischemic hindlimb (ex vivo studies demonstrated cells are in the liver, spleen, and lungs. Day 6: Labeled cells are beginning to appear in the ischemic (left) hindlimb. Days 7 and 11: Peak redistribution of cells to the ischemic hindlimb is event. Control mouse. This mouse had femoral artery ligation but no hMSC injection. No fluorescence is demonstrated at the site of ischemia.

These findings in combination with the following experiments demonstrated that there was a gradual release of the ivMSCs that are acutely entrapped in the lungs and other tissues, which lead to migration and redistribution so that an increased number of human MSCs engraft into ischemic tissue over the several days following intravenous injection, which is efficacious to recovery of ischemic hindlimb muscle. The examples taken together indicate that itMSCs delivered intravenously exerted biologically relevant therapeutic effects on the limbs of subjects with peripheral arterial disease and hindlimb ischemia.

### Example 3

### Intravenous Delivery of Human MSCs Given to Mice with AMI, with Ischemic Cardiomyopathy; MSCs Homing to Ischemic Tissue, MSC Mobilization and Redistribution to Ischemic Tissue from Reservoir Tissues, and Optimization of Administration

I. Distribution of iv administered MSCs that have been grown under hypoxic conditions. Goal: To determine whether iv administration of MSCs results in MSC engraftment into the ischemic tissue.

The rationale for iv administration is the ease and safety of this route, and the fact that, if needed, multiple injections, over time, can be made.

Hypothesis tested: MSCs grown under chronic hypoxia, when injected iv after acute myocardial infarction (occlusion/reperfusion of the LAD), preferentially home to and engraft in ischemic myocardium.

Mouse MI (occlusion/reperfusion) model:
Occlusion (45 min)/reperfusion of the left anterior descending coronary artery (LAD)
iv injection of 1×106 of human itMSCs (CardioCell) 24 hr post MI
Assessment of distribution 24 hr post injection

MSCs utilized:

In all experiments depicted human MSCs (Stemedica) were administered (passage 4) at a dose of 1×106, except the last experiment (MSCs injected 60 day post MI). In this study mouse MSCs were obtained from CD1 mice and cultured continuously in 5% 02. They were administered to CD1 mice, with prior MI, at a concentration of 2×106. to CD1 mice Methods to access tissue Distribution:

1. Radiolabeling of MSCs with indium-111 oxine for:

a) short axis cross-sections for phosphor imaging;

b) gamma counting of the LV

c) In vivo SPECT imaging

2. IVIS ex vivo imaging of heart 24 hr post MSC injection.

3. Flow cytometry.

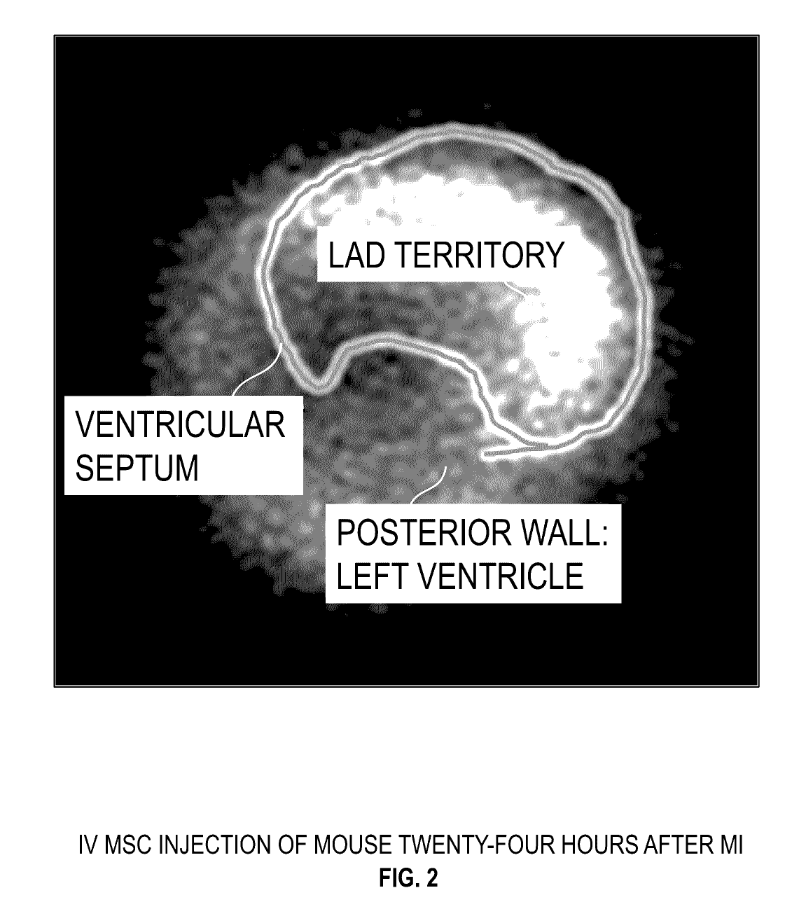

1a. Myocardial Distribution of Radiolabeled hypoxia-grown human MSCs (indium-111 oxine phosphor imaging). FIG. 2 MSC Injection 24 Hours after MI; Imaging 24 h after MSC injection. This figure demonstrates greater signal intensity within the LAD territory, indicating preferential homing and engraftment of MSCs into the region of myocardial injury.

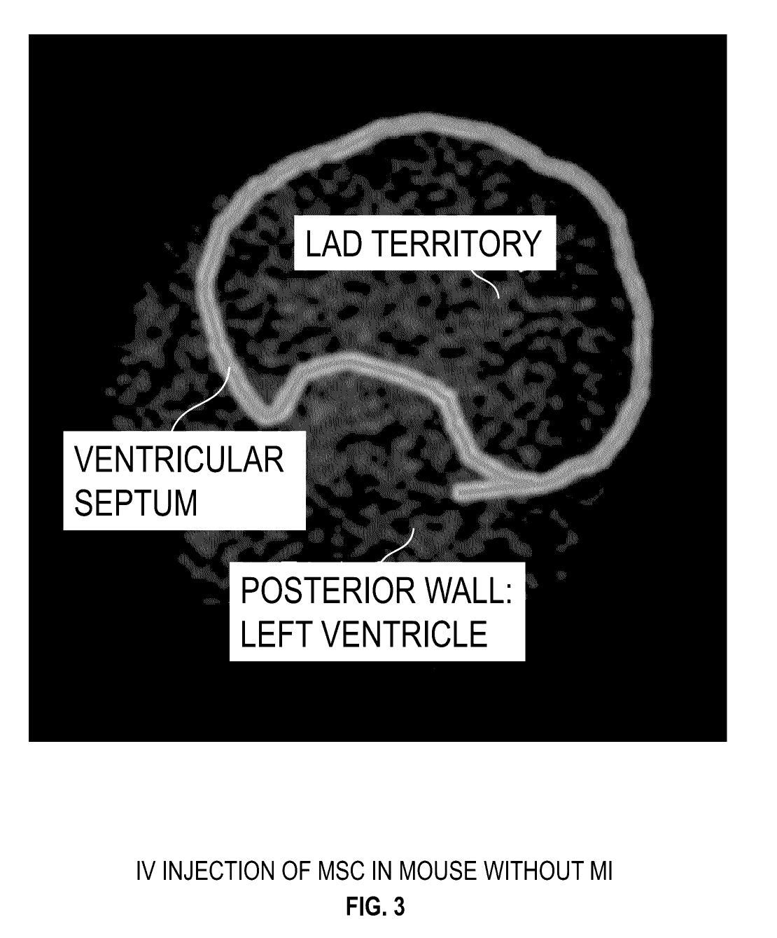

Human MSC injection in mouse with no MI. Imaging 24 h after MSC injection. FIG. 3. This figure demonstrates that iv injection of MSCs in a mouse without a myocardial infarction results in uniform signal intensity throughout the LV wall. This indicates no preferential homing and engraftment of MSCs in the absence of myocardial injury.

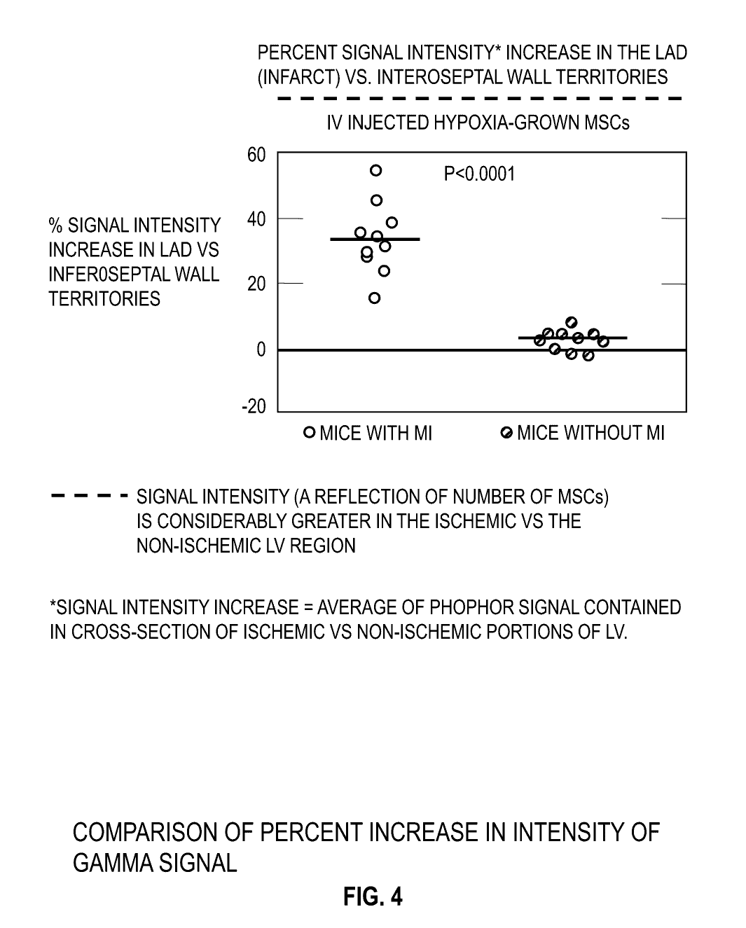

FIG. 4 compares the percent increase in intensity of the gamma signal in the LAD territory vs. non-LAD areas in mice with MI (and therefore with LAD territory ischemic) vs. mice without an MI and therefore without LAD ischemic territory.

Of note, there was considerable mouse-to-mouse variability in signal intensity in mice with MI. We thought this might derive from variability in the amount of ischemic myocardium. We therefore tested the following hypothesis:

Hypothesis: Total gamma signal present in the LV (i.e., the total number of MSCs present) will be significantly influenced by the amount of myocardium infarcted—the greater the infarct the greater the signal.

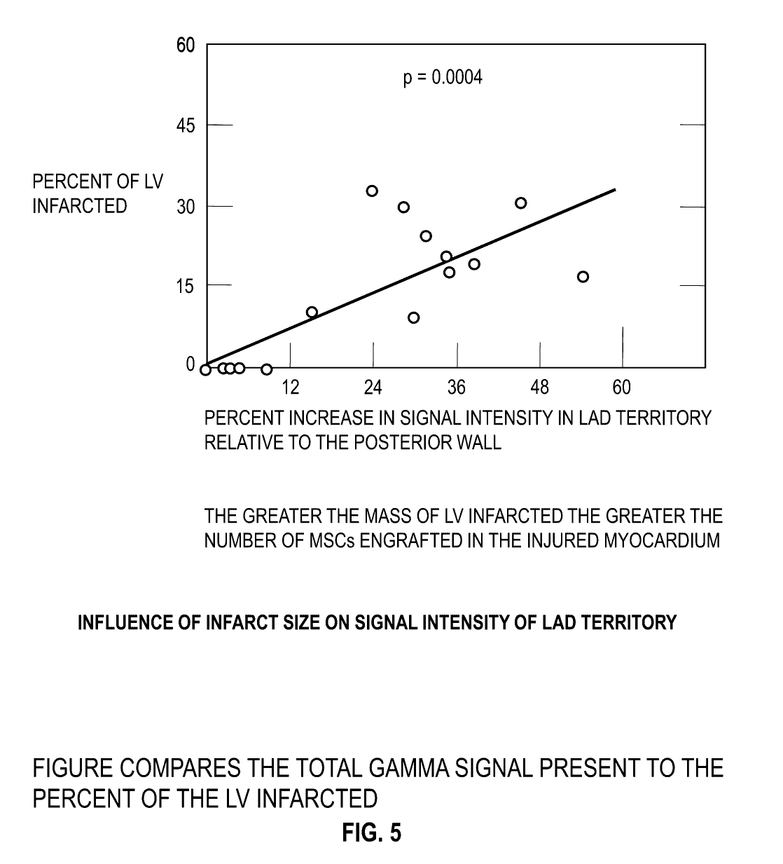

FIG. 5 figure compares, the total gamma signal present to the percent of the LV infarcted.

As expected, there was considerable variability in the amount of myocardium infarcted from mouse to mouse. Importantly, there is a highly significant association between signal intensity and mass of LV infarcted.

This provides powerful proof that MSCs not only home to infarcted myocardium, but they home in proportion to the amount of myocardium infarcted.

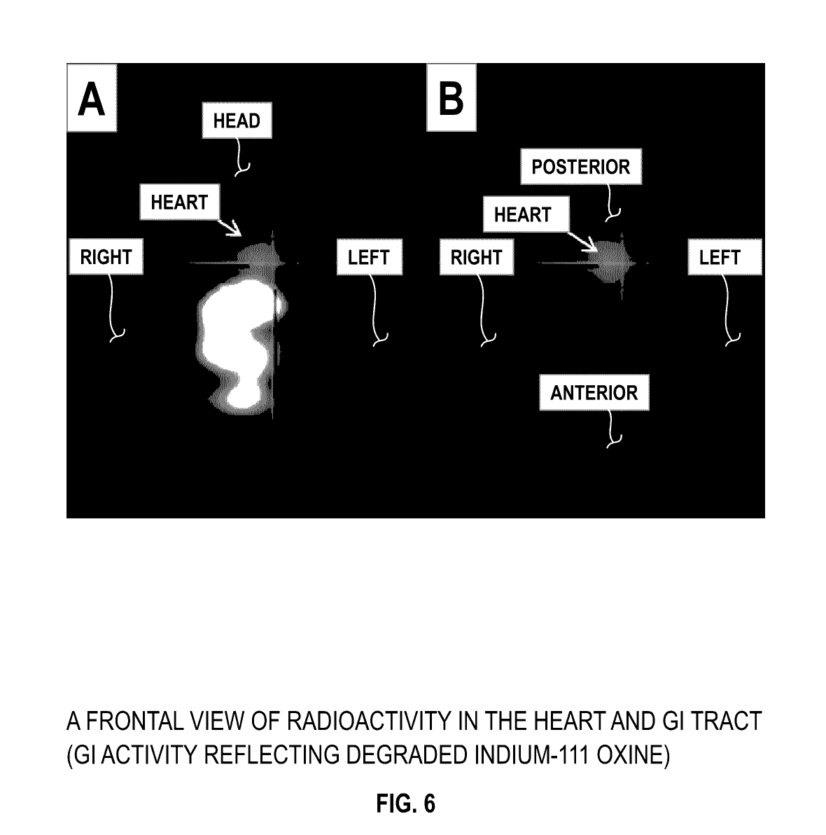

1c. Radiolabeled (indium-111 oxine) hypoxia-grown human MSCs: In vivo SPECT imaging. FIG. 6 shows a frontal view of radioactivity in the heart and GI tract (GI activity reflecting degraded indium-111 oxine). A cross-sectional view at the level of the heart is depicted by a horizontal red line. Panel A shows a frontal view of the mouse. The large very bright area represents GI activity of degraded indium-111 oxine. Heart identified by arrow. Panel B depicts the cross-sectional image of Panel A, with cross-section taken at the level of the horizontal red line. This eliminates the GI track and only includes the heart, as identified by the arrow. This demonstrates, in a living mouse, uptake and engraftment of labeled MSCs. in myocardium of a mouse with AMI.

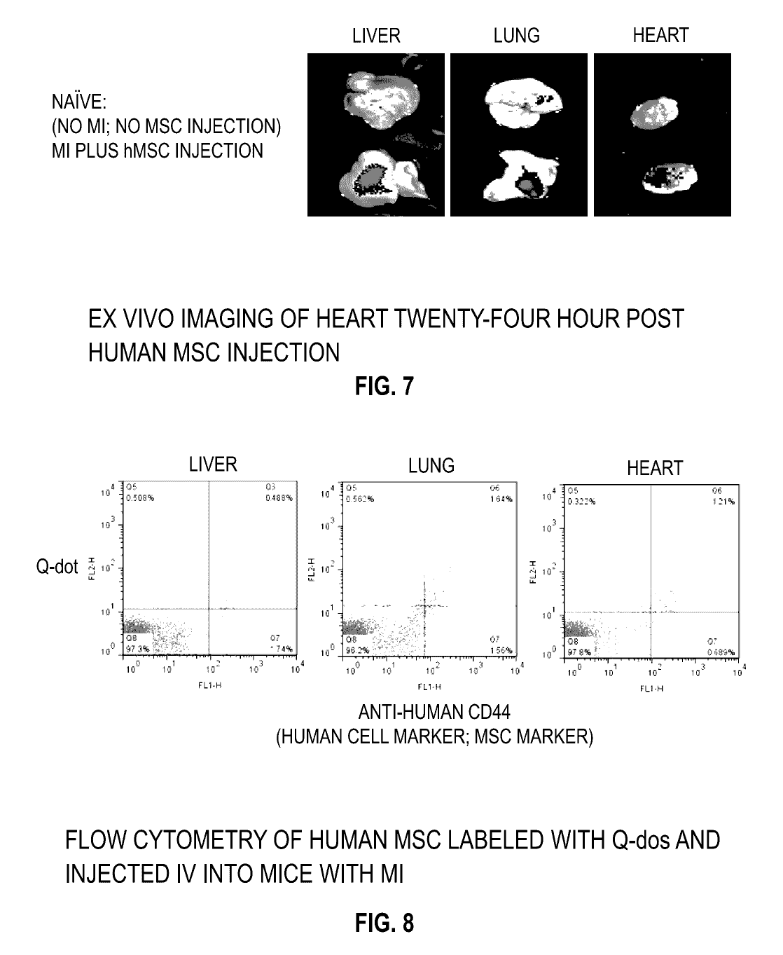

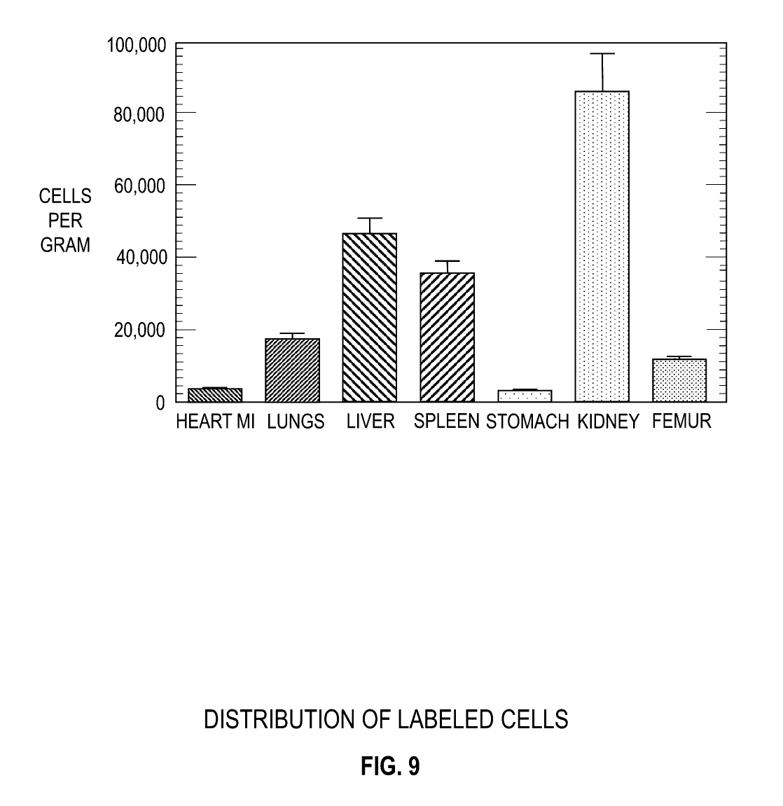

2. IVIS ex vivo imaging of heart 24 hr post human MSC injection. FIG. 7. The in vivo imaging system (IVIS) is a versatile imaging system that uses bioluminescent and fluorescent reporters to identify and semi-quantitate label present in vivo or ex vivo, In this case, we have identified ex vivo tissue localization of iv injected labeled human MSCs. Injection was performed 24 h after MI and tissues harvested 24 h after injection. The images reveal that the iv injected human MSCs home to and engraft in the liver, lung and the heart.

3. Flow cytometry. FIG. 8.

Human MSCs were labeled with Q-dots and injected iv into mice with MI (as above). Flow cytometry was employed to identify MSCs labeled with Q-dots and carrying a cell surface marker specific for human MSCs (CD44). Each individual dot is indicative of an individual cell. Cells in the upper right quadrant carry the Q-dot and the human MSC marker at high intensity, indicating definitively that human MSCs home to the ischemic myocardium.

Cellular distribution of human MSCs, grown under chronic hypoxic conditions and injected 24 h following MI, with mice sacrificed 24 h after MSC injection. [From studies with radiolabeling of MSCs with indium-111 oxine] At 24 h following injection of human MSCs into mice with MI most of the MSCs are located in the kidney, liver, spleen, and lungs. Approximately 1% of the cells are taken up by the myocardium.

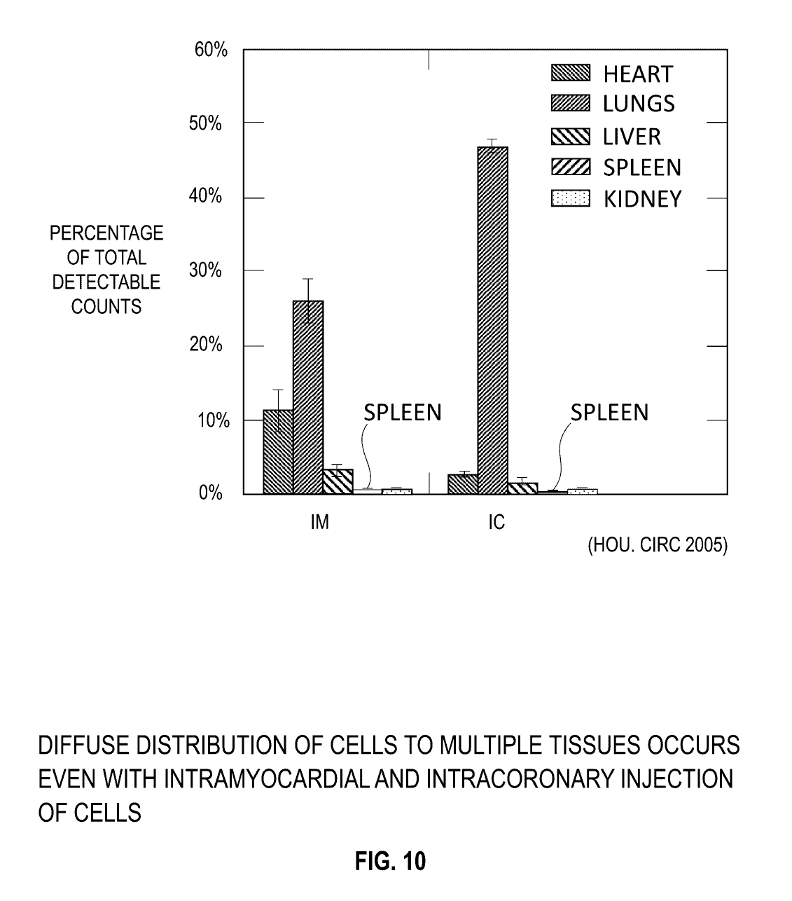

Of note, diffuse distribution of cells to multiple tissues occurs even with intramyocardial and intracoronary injection of cells. This is illustrated in FIG. 10.

Diffuse distribution of cells to multiple tissues occurred even with intramyocardial and intracoronary injection of cells.

IC injection: 5-7 days after coronary occlusion-reperfusion 107 cells (111In-labeled PBMCs) were infused over 30 to 45 sec.

These overall results thus demonstrate that MSCs grown under chronic hypoxia, when injected iv after acute myocardial infarction (occlusion/reperfusion of the LAD), preferentially homed to and engrafted in ischemic myocardium, and that the magnitude of engraftment was directly related to the magnitude of the infarcted myocardium.

The results also lead to two questions:

1. Since any beneficial effects of adult stem cells accrue from paracrine effects, can the 1% of the total MSCs injected that engraft in the myocardium exert biologically relevant beneficial effects—with or without additional effects deriving from the secretion of biologically relevant factors from cells located in other tissues?

2. As suggested in the literature, do the tissues (lungs, liver, spleen) in which the MSCs are located 24 h after injection act as cell reservoirs and gradually release the MSCs with subsequent uptake into ischemic tissue over the next several days? As evidenced above in Example 2, this “redistribution” does in fact occur.

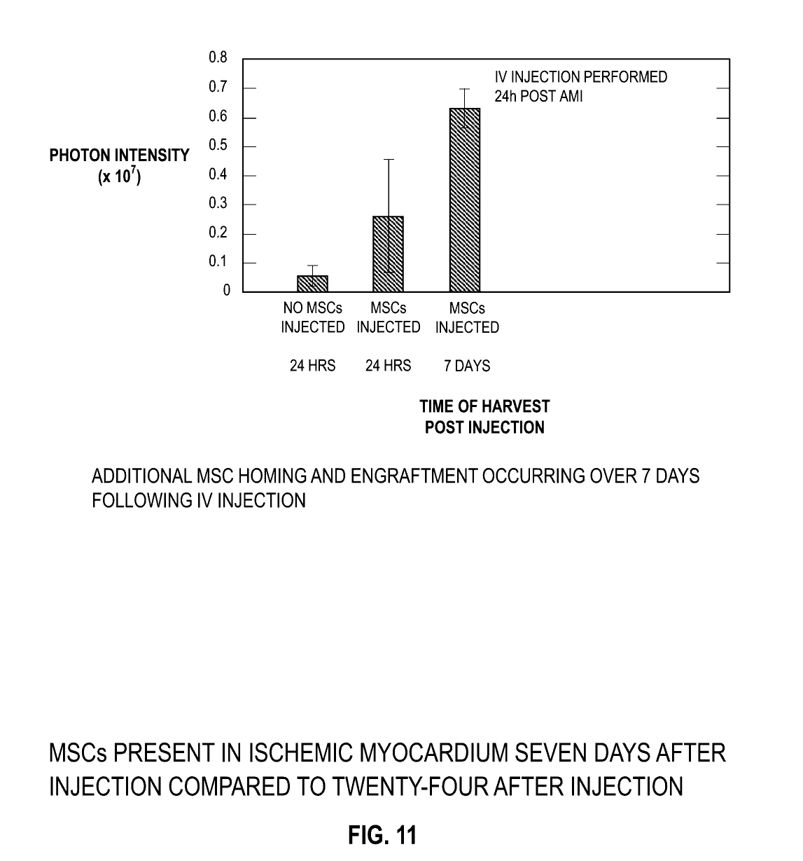

To confirm that a similar gradual redistribution to ischemic myocardium of iv injected MSCs occurs, we determined whether a greater number of MSCs is present in ischemic myocardium 7 days after injection compared to 24 hours after injection. FIG. 11.

Dose=1.5×106 cells/mouse. Assessment of number of cells engrafting in ischemic myocardium was estimated by using Q dot labeling of cells and ex vivo IVIS quantitative imaging.

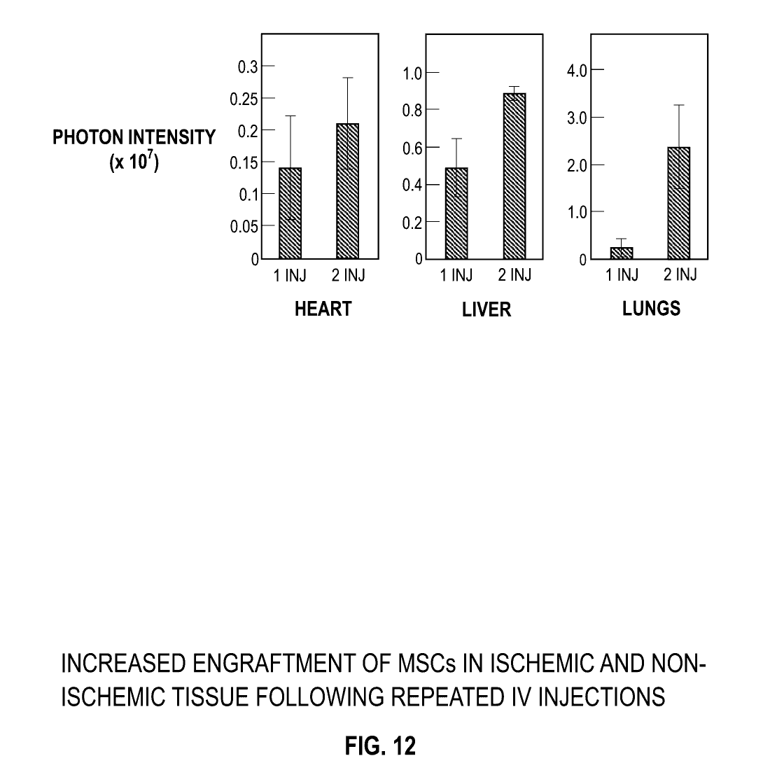

Increased engraftment of MSCs in ischemic and non-ischemic tissue following repeated iv injections. FIG. 12.

We also hypothesized that MSCs injected iv at day 1 and 2 after AMI incrementally engraft into ischemic myocardium (as well as in reservoir tissues) such that the total number of MSCs in the ischemic myocardium substantially increase.

FIG. 12 demonstrates the validity of this hypothesis. We found that two iv injections of MSCs at 24 and 48 h post AMI enhance cell accumulation in the heart, liver and lungs at 7 days post AMI. (Each dose=1.5×106 cells/mouse, n=6)

4. Repeated injections and circulating cytokines.

These findings indicate that MSCs injected iv at day 1, 2, and 3 after MI will lead to incremental increases in plasma VEGF and other cytokines that have been shown to be beneficial to recovery of ischemic tissue.

Conclusions: The multi-dose concept for optimizing the initial dose of MSCs has been validated: Once we determined the toxic dose to a single administration of MSCs, we found that this total dose can be given safely if aliquots of the toxic dose are injected over 1-2 sequential days. Moreover, we found that MSCs injected iv at day 1 and 2 after AMI incrementally engraft into ischemic myocardium (as well as in reservoir tissues) such that the total number of MSCs in the ischemic myocardium and the reservoir tissues is substantially increased by the second dose. We also found that there is a gradual release of the MSCs that are acutely entrapped in the lungs and other tissues, leading to migration and redistribution so that an increased number of human MSCs engraft into ischemic tissue over the several days following injection.

III. Assessment of the viability of human MSCs in the mouse

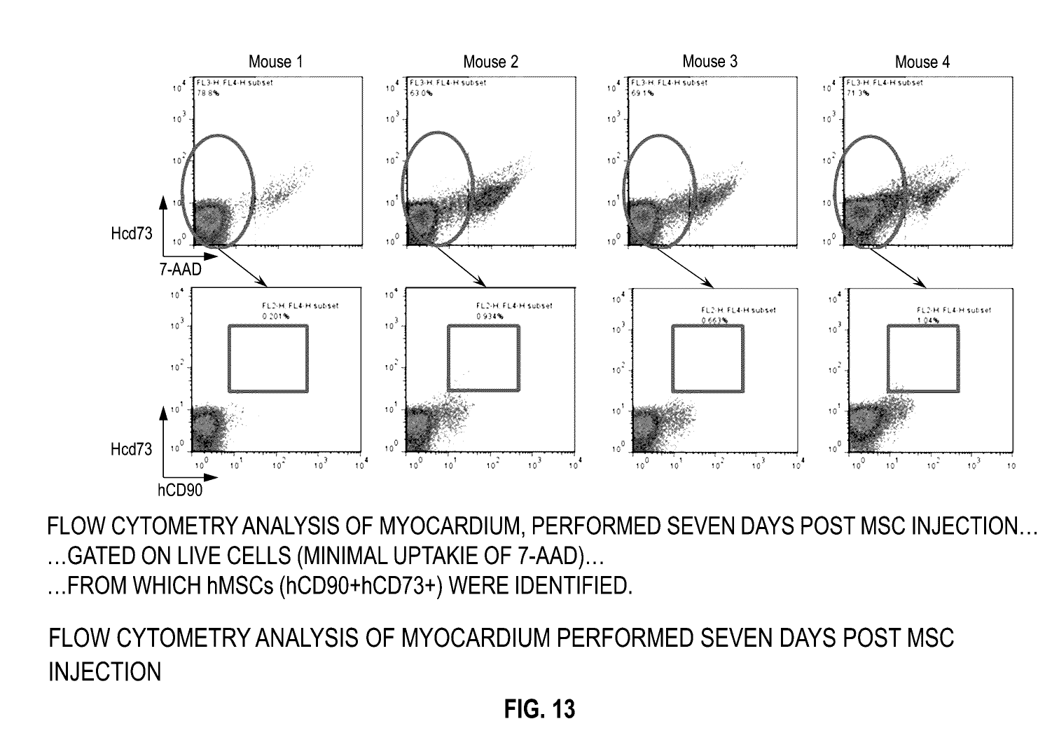

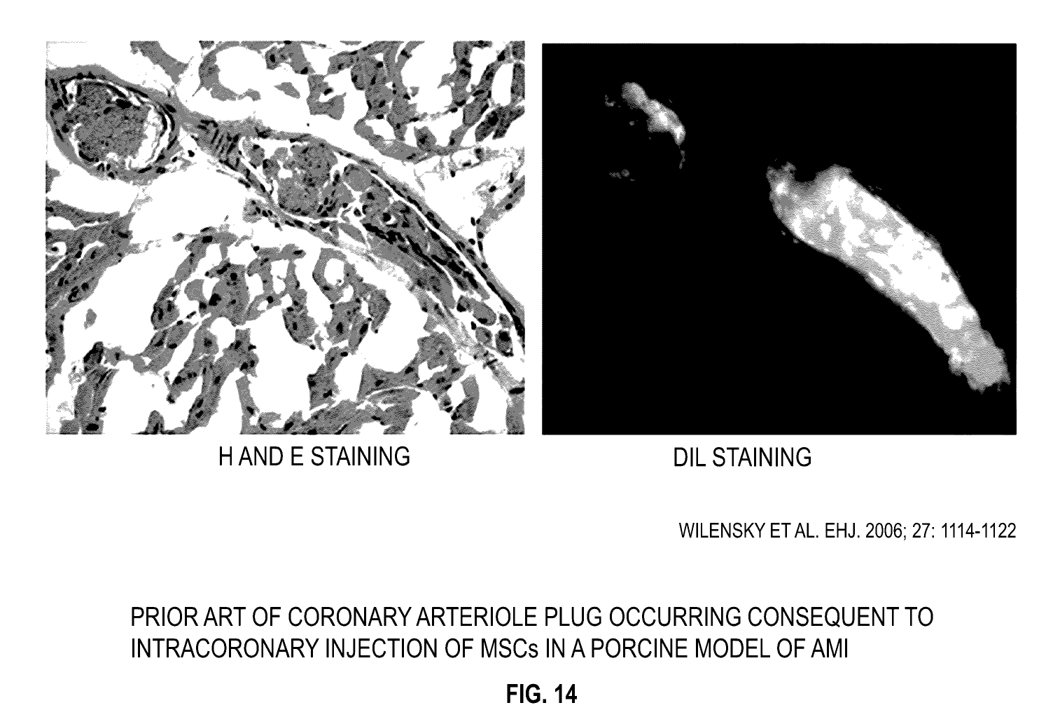

Since viability of human MSCs injected into the mouse is critically related to our future investigations, we performed an additional study to determine whether human MSCs remain viable. FIG. 13 shows a flow cytometry analysis of myocardium performed seven days post MSC injection.

Thus, LAD occlusion/reperfusion was performed and human MSCs, grown under chronic hypoxic conditions (Stemedica's) were injected 24 hours after AMI. Hearts were harvested 7 days later and, using flow cytometry, cells in the heart tissue were sorted by use of a marker identifying live cells (minimal uptake of 7-AAD). Gating was performed on the live cells and then human MSCs were identified using antibodies targeted to hCD90+hCD73+.

This study demonstrated, in 4 different mice, that human MSCs that were administered iv are present in the ischemic myocardium of mice 7 days following injection, and that these cells are still viable.

Conclusion: Human MSCs do remain viable in the ischemic myocardium for at least 7 days after iv injection.

IV. Defining a unique advantage of iv administration of stem cells. One safety-related issue of intracoronary stem cell injection derives from possible thrombotic occlusion of coronary arteries by injected stem cells . . . .

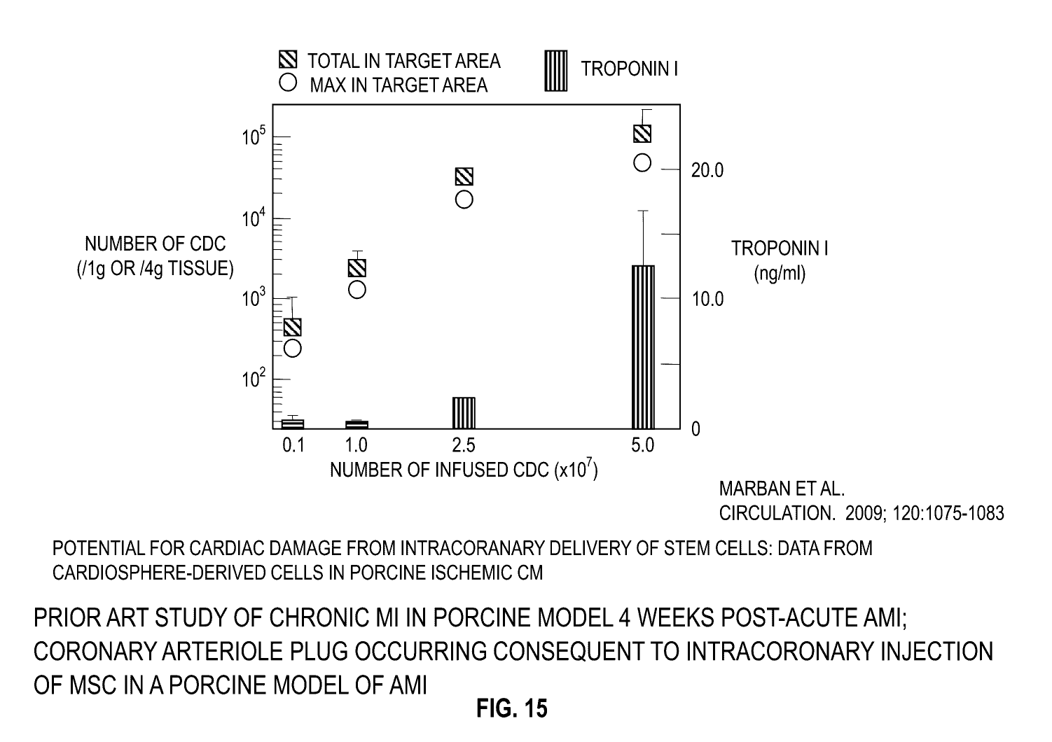

Marban et al (FIG. 15) demonstrated similar findings. They performed studies in a porcine model of chronic MI (4 wks post-acute AMI) and demonstrated that injected CDCs (cardiosphere derived cells) did cause coronary plugging resulting in myocardial injury, as evidenced by increased troponin I levels.

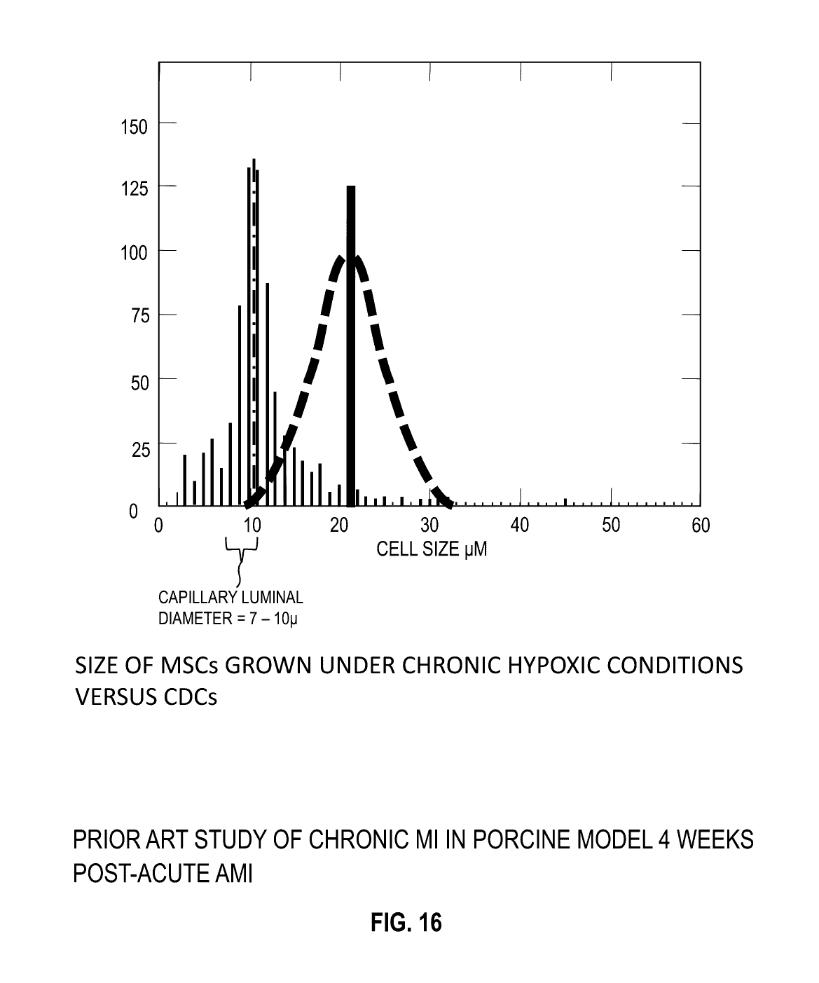

Additional data FIG. 16 from Marban's group indicated that the CDCs were quite large, much larger than the diameter of capillaries. We therefore compared the diameters of Stemedica's MSCs (cultured in chronic hypoxia) vs. the CDCs used by Marban (Capricor). This size factor led to our concept that one advantage of iv administration of stem cells is that the first capillary bed seen by the cells is the pulmonary—which could filter out the larger cells before they could plug arterioles or capillaries of tissues that would be particularly sensitive to the ischemic effects of such plugging, such as the heart and brain.

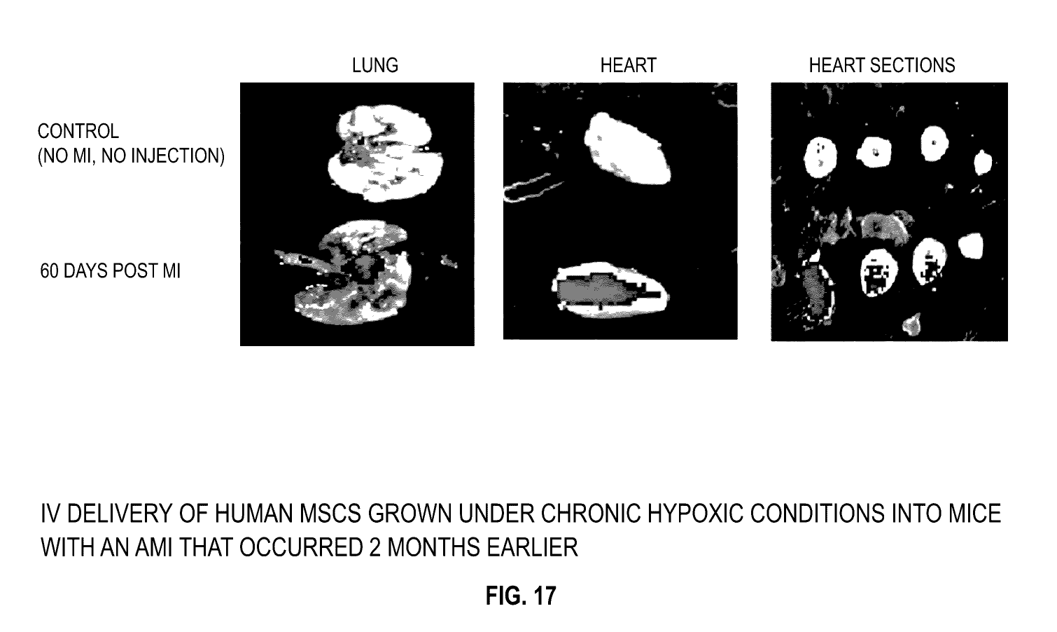

A further study was performed to determine if MSCs grown under chronic hypoxic conditions, home to and engraft in myocardium with old (60 days) MI. FIG. 17. We injected, iv, human MSCs grown under chronic hypoxic conditions into mice with an AMI that occurred 2 months earlier, thus providing a model of chronic ischemia-induced cardiomyopathy. Following are the results obtained from the first mouse so studied.

2×10⁶ mouse MSCs (labeled with Q-dot 525) were injected 60 days post MI, with ex vivo IVIS imaging performed 24 h post-injection. The last panel shows transverse sections of the heart proceeding from base to apex. This study demonstrated myocardial uptake of MSCs even when the acute ischemic event occurred 60 days prior to MSC injection. These findings are consistent with the existence of myocardial injury signals in patients with chronic ischemic HF such that MSCs grown under chronic hypoxic conditions may engraft in such myocardium.

The following studies in humans demonstrated that intravenously administered ischemic tolerant mesenchymal cells distributed and localized to infarcted tissue in the myocardium, supporting the reasonable expectation that intravenously administered ischemic tolerant mesenchymal cells distribute and localize to vasculature conditioned by peripheral arterial disease where the mesenchymal cells have therapeutic efficacy.

### Example 4

### Administration of Ischemic Tolerant Cells

The purpose of this study was to determine the effects of ischemic tolerant mesenchymal stem cells (itMSCs) in patients with acute coronary syndrome (STEMI) with left ventricular systolic dysfunction and ejection fraction ≦45%. Human subjects were selected for a study based on the following inclusion/exclusion criteria:

Trial Inclusion Criteria

Age: less than 60;

STEMI Infarction type in accordance with World Health Organization classification;

Percutaneous coronary intervention (PCI) (coronary angioplasty) performed within 12 hours from the beginning of pain syndrome;

Single-vessel disease with still patent infarct-related artery. LVEF ≦45% post coronary angioplasty

Trial Exclusion Criteria

Past incidence of myocardial infarction;

Cardiomyopathy;

Atrial fibrillation or atrial flutter;

Heart surgery in past;

Critical heart valve disorder;

Disorder of hematopoietic system;

Heart insufficiency type IV functional classification of New York Heart Association (NYHA);

Critical renal, lung or liver disorder, or cancer;

Confirmed damage of more than one of three main coronary arteries;

Intracardiac thrombus; bone marrow disorder.

Day 0: Patients with STEMI, undergone successful percutaneous coronary intervention of artery affected by infarction within 12 hours from inciting event.

Day 1-2: Randomization of patients in two groups, ECG, echocardiogram, collection of blood samples after myocardial infarction. 25 patients were selected and grouped as follows: 10 patients were assigned to an experimental group and 15 patients were assigned the control group.

Day 7: The experimental group received an intravenous injection of about 25-100×106 cells ischemic tolerant MSC from Example 1. The control group received an intravenous injection of saline solution.

Day 14 and 3 Months after MSC or PS administration: ECG, echocardiogram, collection of blood samples

6 Months after MSC or PS administration: ECG, echocardiogram, collection of blood samples

1 Year after MSC or PS administration: ECG, echocardiogram, collection of blood samples.

Results

TABLE 1

| | Experimental | | |
|---|---|---|---|
| Patient data | group | Control group | P |
| Patient number | 19 | 15 | |
| Average age | 51.4 ± 3.5 | 52.7 ± 6.1 | Not-signif |
| Average time for | 5.7 ± 2.5 | 4.5 ± 3.7 | Not signif |
| recanelization of infarct | | | |
| related artery, hours | | | |
| % occlusion of infarct related | 79/14/7 | 71/21/8 | Not signif |
| artery: anterior descending | | | |
| artery (PNA)/right coronary | | | |
| artery (PCA)/circumflex | | | |
| artery, % | | | |
| Degree of acute failure in | 19/45/34/2 | 24/42/35 | Not signif |
| accordance with Killip | | | |
| functional classification | | | |
| I/II/III/IV, % | | | |
| Presence of preinfarction | 46 | 53 | Not signif |
| angina, % | | | |
| Early postinfarction angina, % | 54 | 48 | Not signif |

TABLE 2

| | Experimental group | | | | |
|---|---|---|---|---|---|
| | | 2 | Control group | | |
| | 1 | After 3 | 3 | 4 | P for |
| Data | Initially | months | Initially | After 3 months | 2 and 4 |
| High sensitive C- | 25.3 ± 7.1 | 3.3 ± 1.5* | 28.7 ± 35 | 13.4 ± 7.3* | <0.001 |
| reactive protein, | | | | | |
| mg/ml | | | | | |
| BNP protein, ng/ml | 862.6 ± 123.5 | 119.2 ± 35.7* | 998 ± 113.7 | 1451 ± 212.8 | <0.001 |
| End-diastolic | 146.4 ± 13.3 | 115.9 ± 21.4* | 137.9 ± 33.1 | 143 ± 53.9 | >0.05 |
| volume (EDV) of | | | | | |
| left ventricle (LV), | | | | | |
| ml | | | | | |
| End-systolic volume | 69.2 ± 8.6 | 46.7 ± 6.3* | 66.9 ± 9.1 | 75.6 ± 11.5 | <0.05 |
| (ESV) of LV, ml | | | | | |
| Quantity of | 8.2 ± 2.9 | 2.6 ± 1.1* | 7.9 ± 3.5 | 5.6 ± 2.2 | >0.05 |
| asynergic segments | | | | | |
| of LV | | | | | |
| Ejection fraction of | 42.1 ± 6.1 | 57.5 ± 3.3* | 46.9 ± 7.1 | 45.5 ± 6.7 | <0.05 |
| LV, % | | | | | |
| Functional class of | | 1.5 ± 0.7 | | 3.1 ± 0.3 | <0.05 |
| chronic heart failure | | | | | |
| in accordance with | | | | | |
| (NYHA) | | | | | |

Administration of itMSC resulted in: statistically significant decrease in inflammation as judged by the level of C-reactive protein; in significant decrease in end-systolic and end-diastolic volume of left ventricle, as well as significant increase in the LVEF from 38.4% to 52.3% at three months and to 54.7% at six months post-administration, which brought his parameter to what is considered to be a normal range for healthy individuals (50-65%).

Combination myocardial revascularization with MSC administration in patients with Acute Myocardial Infarction resulted in improvement of overall and local contractive myocardium functions and also normalization of systolic and diastolic filling of left ventricle.

Intravascular Treatment of Rats with Mesenchymal Stem Cells for Repair of Heart Tissue After Ischemic Damage

A widely used and attainable model of a heart attack induced on laboratory rats is the method of coronary occlusion (Skrikanth G V N, 2009).

Methods:

Experiments were carried out on white male outbred rats weighing 190-200 g. according to the general ethical principles of experimentation on animals within the agreement of the European Convention regarding the protection of vertebrates for experiments or other scientific purposes (2003).

The animals were divided into the following groups: control, n=12; rats falsely operated on, (FO) n=10; a group suffering from untreated myocardial infarction (MI), n=14; a group which suffered myocardial infarction which received cellular therapy (CT), n=11; and a group which suffered myocardial infarction and received cellular factors (CF), n=12. All of the substances were administered in sterile conditions using an insulin syringe injecting into the tail vein.

On the third day after the receipt of electrocardiographic data on the occurrence of an acute heart attack, the IM group received 4 injections of a physiological solution (0.5 ml EOD), the SC group received a single injection of a stem cells in suspension (3 mln./subject), the CF group received 4 injections of cell factors (0.5 ml EOD).

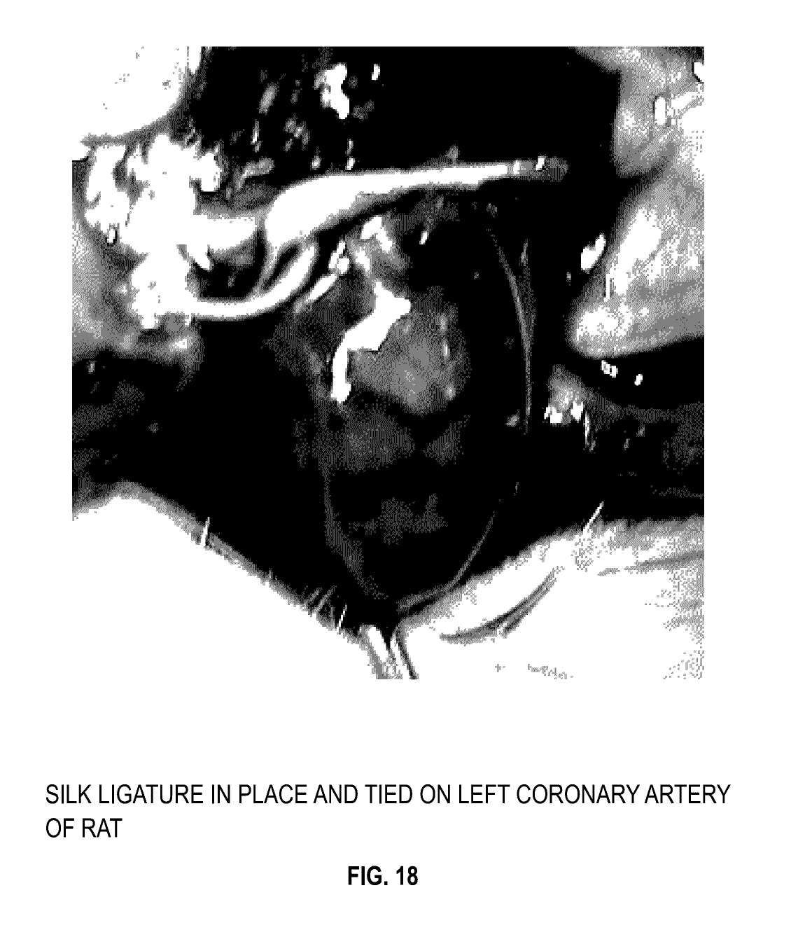

Surgical manipulation was performed in sterile conditions under anesthesia. Myocardial infarction of the experimental animals was modeled by ligation of the descending branch of irreversible left coronary artery (LCA). For this procedure the rats' skin was opened on the left side of the rib cage and the pectoral muscles were separated in a bloodless way to expose the chest wall. The chest was opened by making an incision the intercostal muscles in the 4th intercostal space between the ribs and separating the ribs with a retractor. Then, using tweezers pericardium was removed. The heart was then carefully removed from the cavity. Under the descending branch of the left coronary artery using an atraumatic needle (5-0) a silk ligature was placed and tied. Tightening the ligature site stopped myocardial blood flow and caused the formation of a cyanostic spot on the surface of the heart. FIG. 18 shows the heart with the placed ligature.

Coronary artery ligation was performed without subsequent reperfusion. In the group of falsely operated animals only a ligature was placed under the artery without making ligations. The heart was then returned to the chest cavity and connected to the ribs. To avoid a pneumothorax, air was removed from the chest cavity, and pressure was increased in the subpleural space by slightly applying pressure on the chest wall. After surgery, the wound was sutured in layers. Cefazolin was subcutaneously administered and the skin was treated with iodine. During the course of the experiment we observed weight gain in the rats.

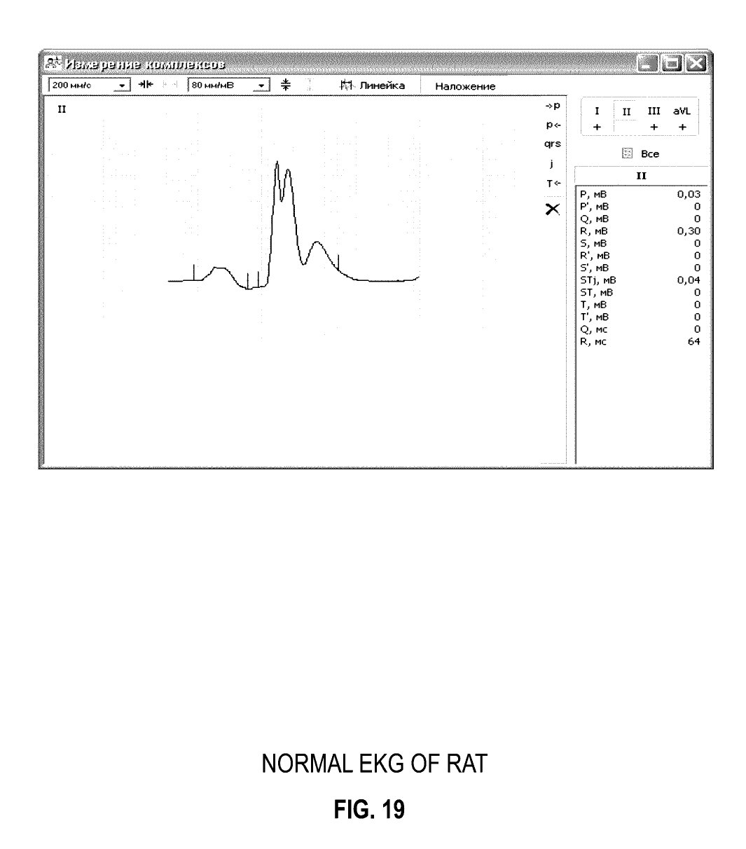

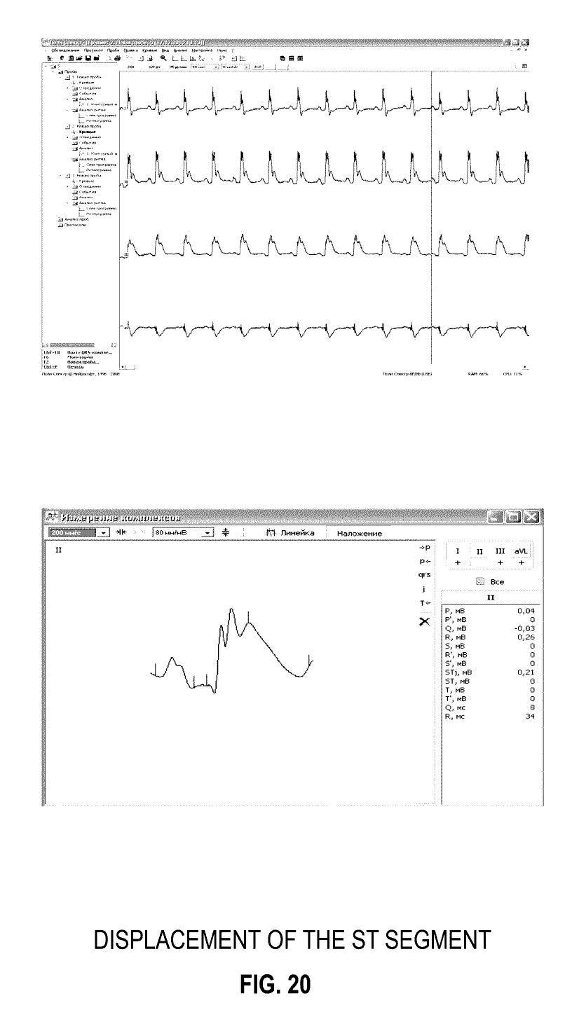

The development of experimental acute myocardial infarction during the experiment was confirmed electrocardiographically, the study was carried out under anesthesia. 1 hour after occlusion and on the 3rd day after occlusion signs of myocardial damage were revealed on the EKG. A computerized electrocardiograph “Polyspectrum-8/B’ was used to record the EKG and, needle electrodes were injected subcutaneously in the distal portions of the 4 limbs. EKG changes were most informative in the rat II standard lead. See FIGS. 18 and 19, normal EKG reading in rats which lack a Q wave. A normal EKG reading in rats lacks a Q wave. (FIGS. 19 and 20). FIG. 19 the averaged shape of a healthy heart EKG in rats. Here and further standard II type of connection is presented. FIG. 19 EKG of a healthy rat. Top to bottom: leads I, II, III, and a VL.

An hour following coronary arterial occlusion, pronounced electrocardiographic changes developed in the rats. At first, there was expansion of and increase in the amplitude of the T wave, indicating a disruption of repolarization which was extremely sensitive to electrical shortages, and the development of significant ischemic damage in the myocardium. Furthermore, deeper lesions were observed indicating the disruption of the process of myocardial depolarization, which was manifested in the displacement of ST-segment above the contour lines (FIG. 20 Displacement of the ST segment.)

FIG. 21 shows the acute stage of myocardial infarction when the high ST segment merges with the increased positive T wave forming a monophasic curve. These are EKGs of rats in the acute stage of myocardial infarction.

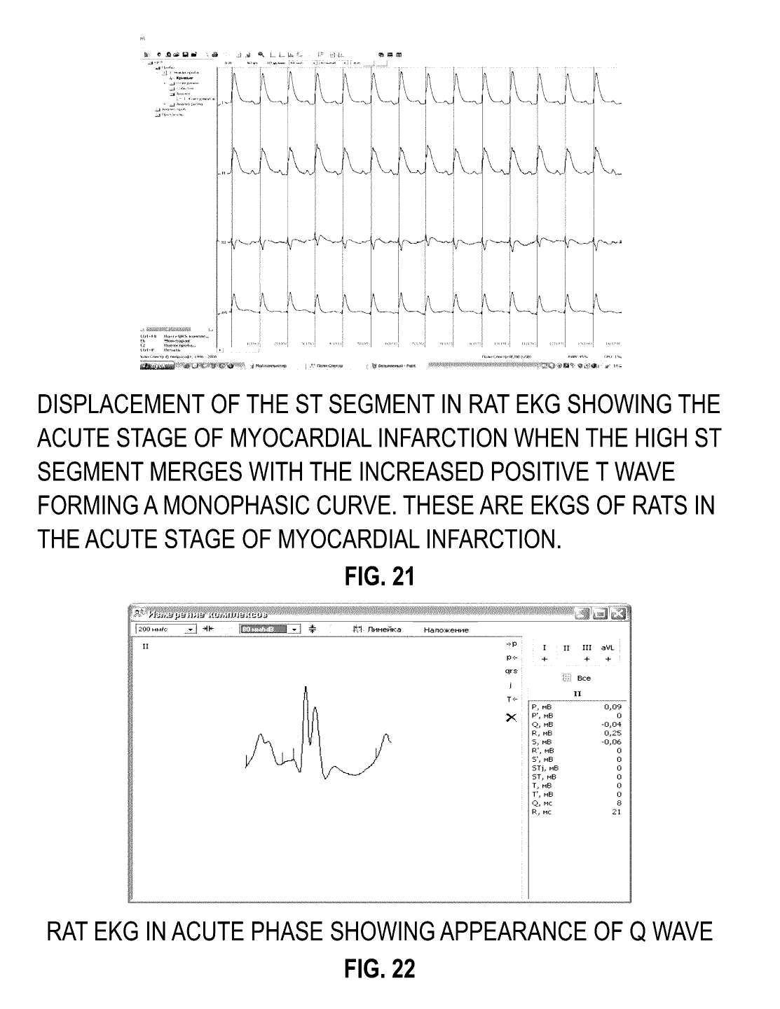

In the acute phase, the EKG of the animals showed the appearance of the Q wave followed by an increase in its depth and a simultaneous reduction in the height of the R wave (FIG. 22), and the appearance of a pathological Q wave.

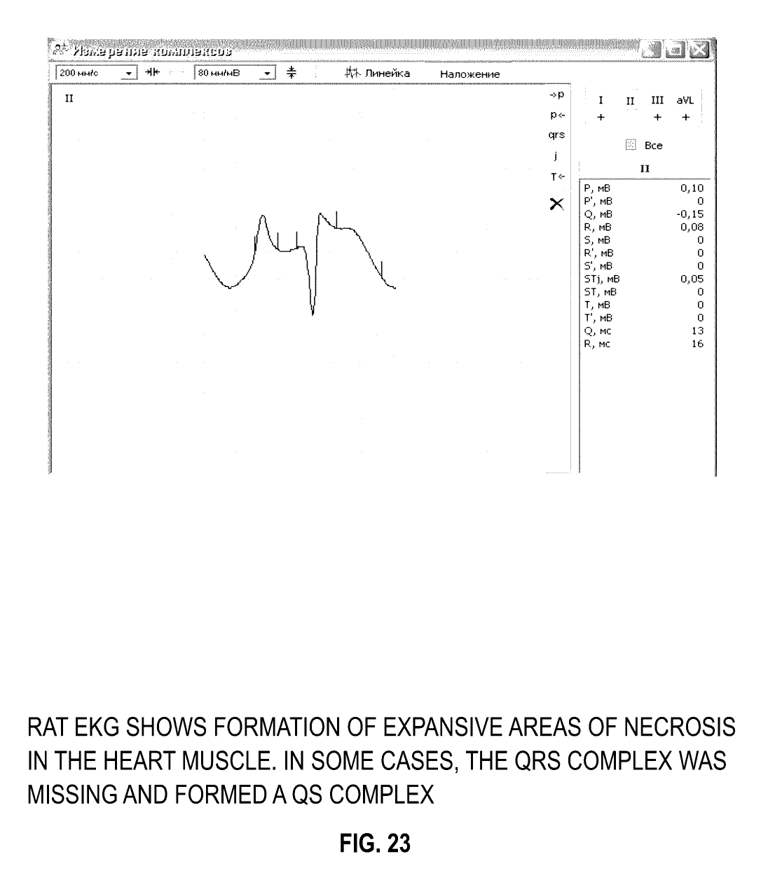

The observed changes of the QRS complex reflect the formation of expansive areas of necrosis in the heart muscle. In some cases, the QRS complex was missing and formed a QS complex (FIG. 23, QS complex).

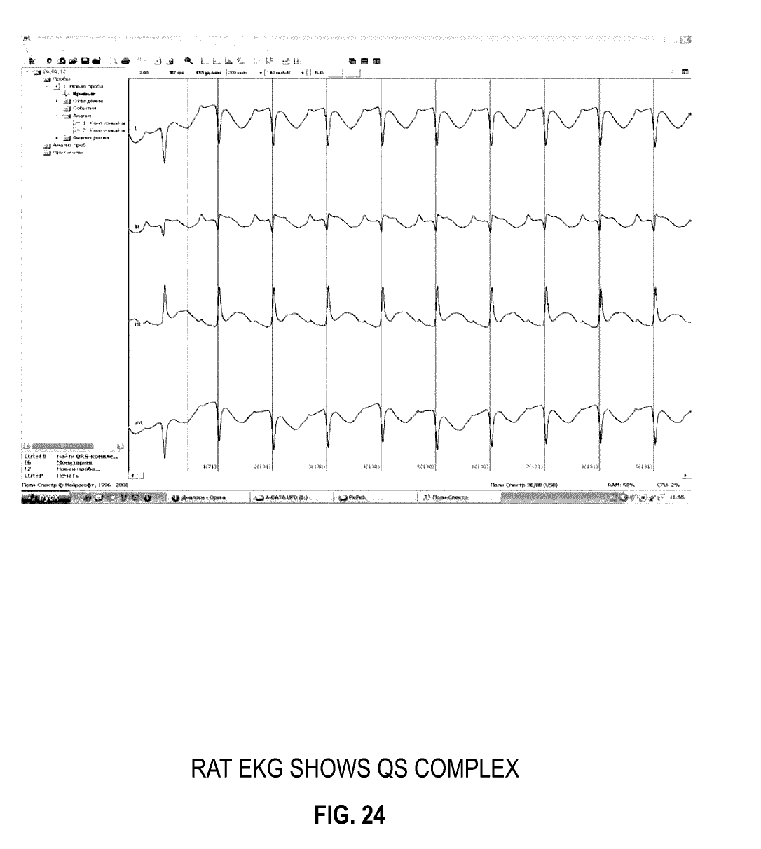

In the following experiment, we took only rats which on the third day after myocardial infarction showed signs of necrosis of the myocardium. On the II standard lead of the EKG: a deep Q wave or a QS complex (FIG. 24) in the EKG of the rats on the third day following occlusion LCA.

A deep Q wave persisted on the 14th day following myocardial infarction. The dynamic of the height of the R wave tended to be lower, and not within the normal range. In the scarring stage of myocardial infarction, strong connective scar tissue was formed on the site where there was necrosis.

Furthermore, the levels of C-reactive protein (CRP) and cerebral natriuretic peptide (BNP) were used biochemical markers of myocardial damage. The markers were determined by an ELISA assay of blood drawn intravenously 14 days after coronary occlusion. Clinical studies show that a high level of concentration of C-reactive protein is associated with a significant risk of death in patients after an acute coronary event. Synthesis of BNP in heart failure increases dramatically, and it is regarded by doctors as a marker in assessing the contractile ability of the heart muscle, and predicting the course of disease. Currently, it has been proven that there is a close relationship between the severity of acute damage of the heart muscle, especially the left ventricle, and the content of BNP in plasma.

CRP and BNP levels in serum were determined by ELISA with a kit from BD Biosciences and Ray Biotech, Inc. Respectively. Definitions and calibration standards were carried out in two parallel dimensions in accordance with the instructions of the manufacturer.

On the 14th day of the experiment, the rats were euthanized with a lethal dose of anesthesia in accordance with the ethical guidelines for removing animals from experiments (1985).

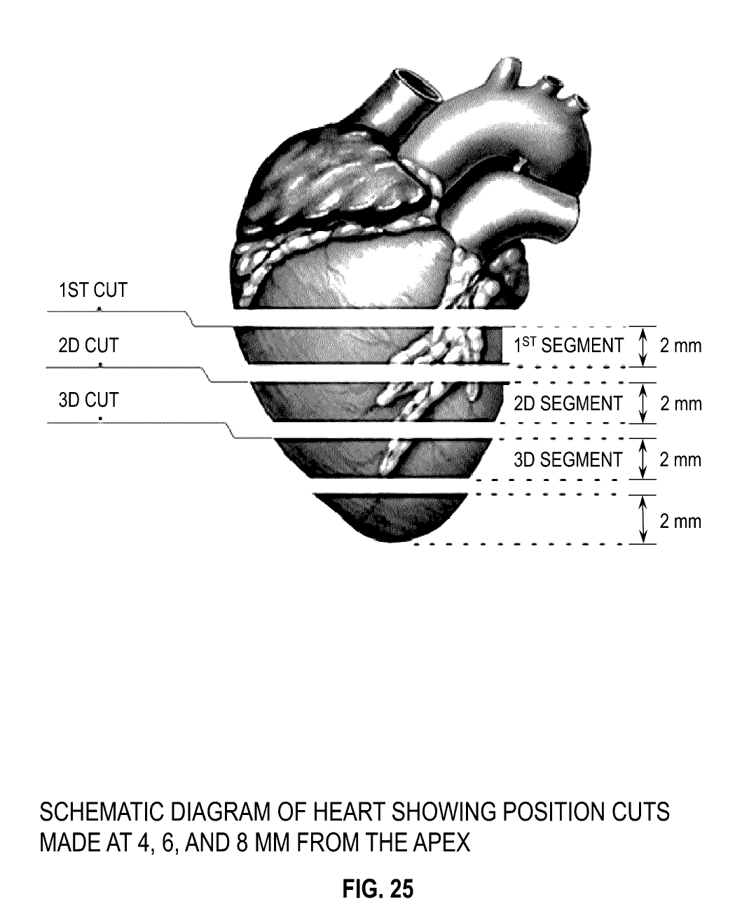

After the rats were euthanized, autopsies were carried out and their hearts were removed for further histological research. The hearts were placed in a 10% aqueous solution of neutral formalin and further filled in paraffin prepared for a series of cross sectional cuts of 5 micrometers on a microtome MHC-2 using standard methods (R. Lalli, 1969; G. A. Merkulov 1969). Cuts were made at 4, 6 and 8 mm from the apex of the heart (FIG. 25), and stained with hematoxylin and eosin for the purpose of evaluation of connective myocardial scar tissue on sight using the Masson Method.

All the material was examined using a ScienOp BP-20 microscope by magnifying the eyepiece 7×, 10× and objectives 4×, 10× and 40×. The material was photographed with a digital camera eyepiece for the microscope-DCM500 (500 pixels, USB2.0).

The intensity of the histological changes was evaluated 14 days after occlusion of the descending branch of the left coronary artery. All histological studies were performed as a double-blind study.

This took into account both the qualitative and the quantitative assessment of structural changes in the center of the infarction and in the peri-infarction zone in the area of the scar tissue. Morphometry was performed using the Image J program of the National Institute of Health (USA) with a set of modules for medical morphometry devised by Wayne Rasband.

The following morphometric parameters were taken and used as the criteria for the evaluation of the functional morphology of the myocardium: the length and breadth of a heart attack, dilatation of the heart, the bulk density of the necrotic myocardium, leukocyte infiltration, functioning myocardium, and the connective tissue, as well as areas of necrosis, infiltration, functioning myocardium, and the connective scar tissue. All bulk densities were calculated by point calculation using an ocular stereometric grid, Avtandilov (1990, 2002).

Given the high variability of the sizes of the necrotic zone in myocardial cross-sections at 6 and 8 mm from the apex of the heart, the comparison of the magnitude of necrotic tissue damage and the area of connective scar tissue was performed on sections at 4-5 mm from the tip. (B. V. Dubovik, 2005)

Statistical analyses were conducted using the software package Statistics 6.0.

Results

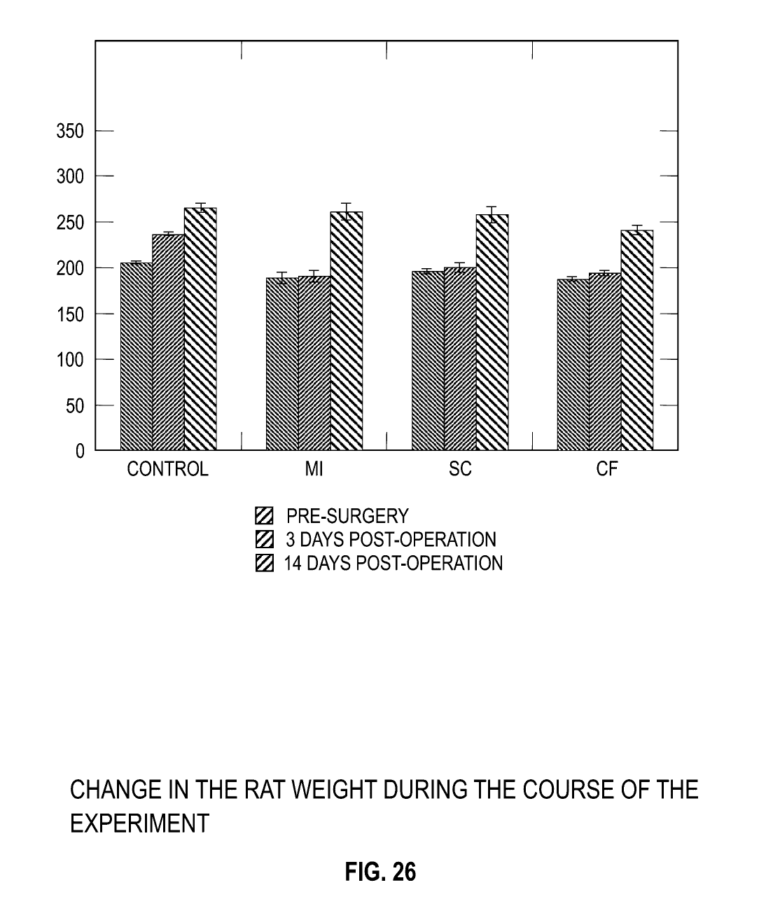

One of the complex parameters which objectively characterized the condition of the body during the experiment as a whole, was the change in the body weight of the rats. This diagram shows that the control group of rats was steadily gaining weight throughout the experiment. All other groups of rats with occlusion of the left main coronary artery: MI, SC, and CF experienced a statistically significant reduction in weight gain on the third day after surgery, 19.7%, 15.5%, 18.04% respectively. This may be due to myocardial infarction. After 2 weeks, the body weight of almost all the experimental groups with myocardial infarction reached the weight of the control group, indicating metabolic recovery by day 14 after undergoing surgery. FIG. 26 shows change in the rat weight during the course of the experiment.

| | | 3 days post | |
|---|---|---|---|
| Group | pre surgery | operation | 14 days post operation |
| Control | 205.20 ± 1.50 | 236.60 ± 1.24 | 265.00 ± 4.75 |
| MI | 188.46 ± 5.25 | 190.00 ± 5.61 | 260.83 ± 9.04 |
| SC | 195.57 ± 2.05 | 199.93 ± 4.77 | 257.92 ± 8.57 |
| CF | 187.47 ± 1.89 | 193.67 ± 2.71 | 240.71 ± 4.62 |

The rats in the CF group gained slightly less weight in comparison to the control group.

The condition of the heart tissue was assessed in photographs taken under the microscope. The size of the zone of damage after coronary occlusion was evaluated in the photographs and the level of dilation of the heart cavities was qualitatively measured as well as the area of working myocardium and the size of the scar tissue.

Analysis of the results of histological examination will begin with a description of the morphological characteristics of the myocardium of the control group

Control Group.

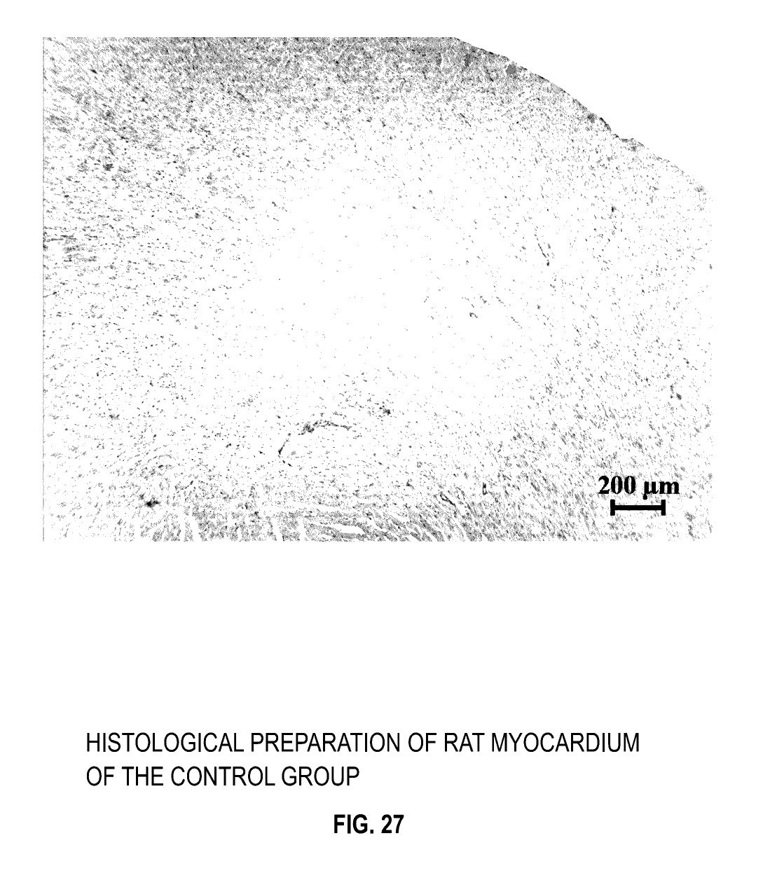

On the histological preparation of the control group the myocardium consisted of striated cardiac muscle tissue (FIG. 27), consisting of anastomosing muscle fibers—cardiomyocytes. Myocytes are clusters of approximately the same thickness and are elongated—rectangular in shape, with clear contours. The oval-elongated nucleus located in the center of the cell is held in place by oxiphylic cytoplasm, which has a distinct longitudinal and transverse striations. There are small coronary artery walls within the sections which are practically unchanged. The endothelium of the blood vessels has a flattened shape and is undamaged. The morphology of the myocardium of the left and right ventricle is not significantly different. Histological section of a healthy myocardial longitudinal section of muscle fibers stauned wih hematoxylin and eosin. Ob.4×Ok.10. Control.

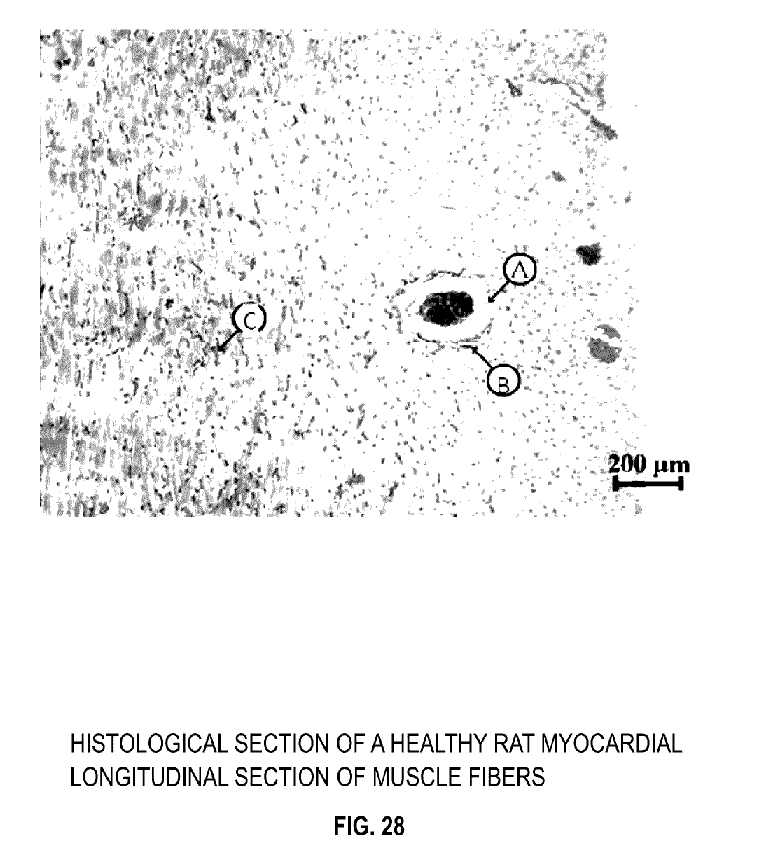

Staining by the Mason Method reveals collagen fibers in a small quantity in the blood vessels in the sub endocardial and sub epicardial layers. FIG. 28 consists of a histological section of a healthy myocardial longitudinal section of muscle fibers. Staining of the connective tissue via the Masson Method. A—artery b—in blue adventitial collagen fibers, B—capillaries. 10×.10. Control.

MI group.

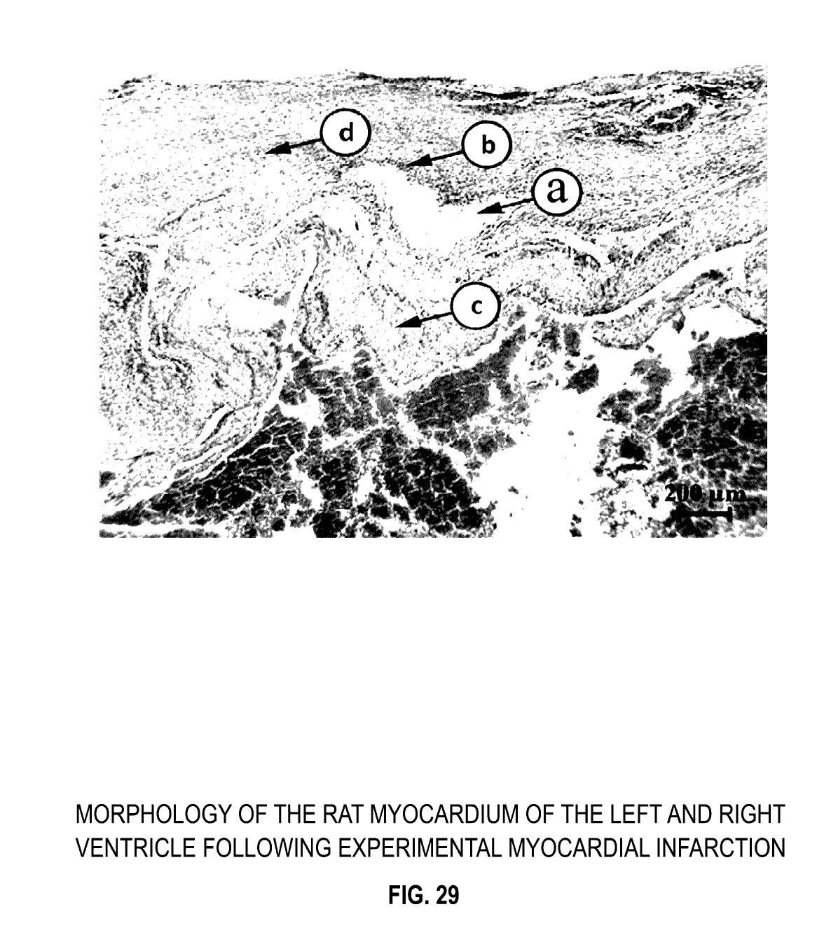

In the next stage morphological characteristics of myocardial infarction was analyzed in rats with no treatment post-surgery. On the 14th day after coronary occlusion the histological preparation of the heart wall is represented by three well-distinguishable layers: the inner (endocardium), medium (myocardium) and outer (epicardium). Morphology of the myocardium of the left and right ventricle is different. In the left ventricle, an area of myocardial infarction is observed. It consists of three well-defined components: the area of cardiomyocyte necrosis (FIG. 29, a), leukocyte infiltration, and young, undeveloped, loose fibrous connective tissue in a state of maturation (FIG. 29, d). Distinct coagulation necrosis of cardiomyocytes is presented in sections as a single focus irregular round shape with clear boundaries. Group MI. Myocardial (v×4, approx. ×10): a—necrotic cardiomyocytes, b—leukocyte infiltration, in B—cardiomyocytes in a state of hypertrophy d—connective scar tissue. Stained with hematoxylin and eosin.

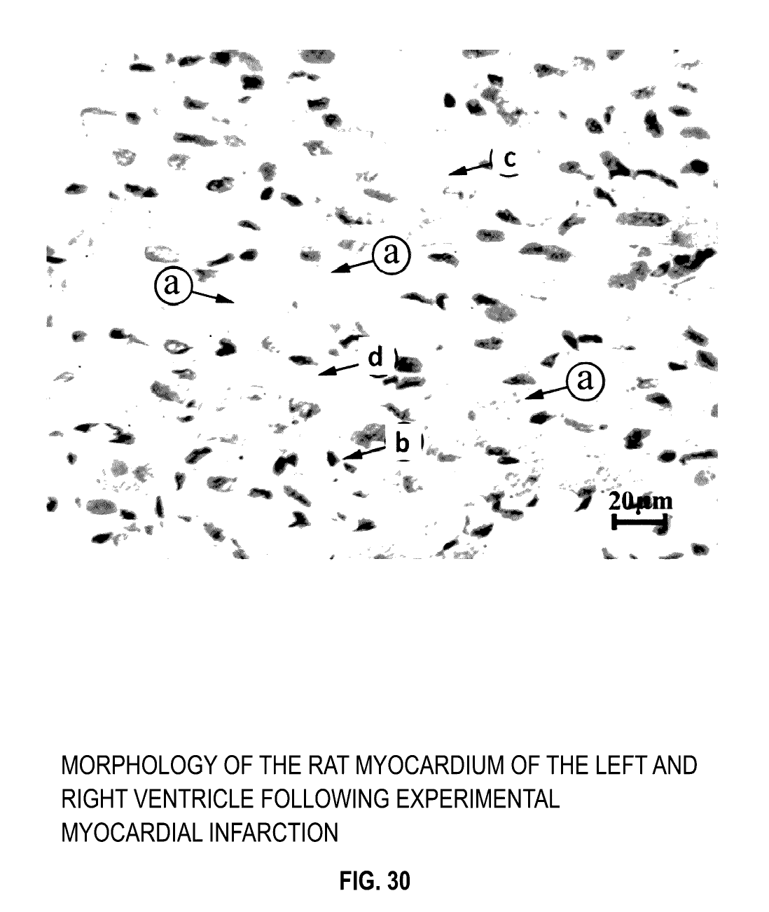

Cytoplasm of cardiomyocytes in the center of necrosis, in light pink the nucleus in a state of karyolysis (FIG. 30,a), part of the fragmented cell (FIG. 30,B). Group MI. Cardiomyocytes (v×40, ca. ×10): a—a cardiomyocyte karyolysis b—white blood cells, and in B—the fragmentation of cardiomyocytes d—granular dystrophy of cardiomyocytes. Staining with Hematoxylin and Eosin.

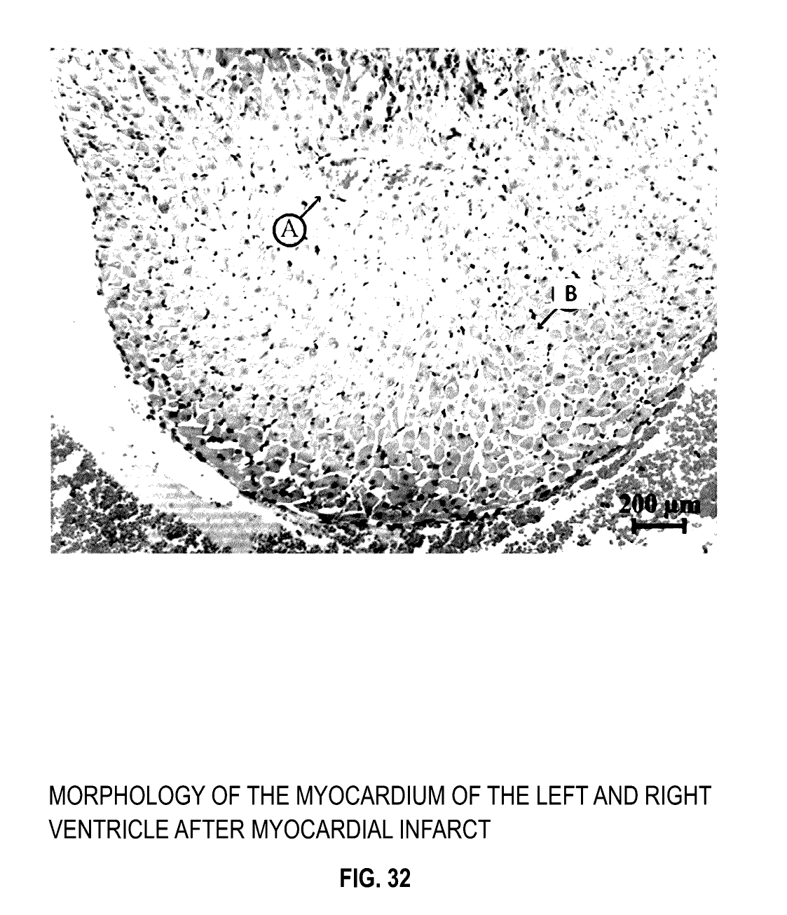

Demarcation inflammation with infiltration of the surrounding tissue by neutrophils and individual macrophages are observed in the area of necrosis (FIG. 30, b). Bundles of muscle fibers adjacent to the site of infarction in this area are thinned, there is pronounced swelling of the intermuscular stroma and strongly diffused leukocyte infiltration (FIG. 32c). Group MI. Myocardium (v×10, approx. ×10): a—a wavy deformation of cardiomyocytes, b—capillaries in a state of increased blood flow, B-leukocyte infiltration. Stained with ematoxylin and eosin.

A wider area is located on the line between demarcation inflammation and healthy cardiomyocytes showing granulated tissue at the stage of maturation.

Irregular morphology of the cardiomyocytes with two alternating patterns of pathological processes is expressed in the protein granular dystrophy of the cardiomyocytes of the right ventricle and the subtotal of their moderate hypertrophy. Dystrophic changes of the cardiomyocytes are seen as bundles of approximately the same thickness, forming an elongated rectangular shape with indistinct outlines of the nucleus which occupies the center of the cell and is held in place by granular cytoplasm with indistinct longitudinal and transverse striations.

Separate cardiomyocytes are in a state of necrosis and necrobiosis which manifests itself as karyolysis. Hypertrophied cardiomyocytes differ in their large size (FIG. 30b and FIG. 32b), uneven fiber thickness, and polymorphism of the nucleus, where some of the cardiomyocytes are round and others elongated. FIG. 32 Group MI. Histological section of the myocardium with a cross-section of muscle fibers, with predominantly moderate-severe hypertrophy of cardiomyocytes. Stained with hematoxylin and eosin. A artery, b—hypertrophic cardiomyocytes. Ob.4×Ok.10.

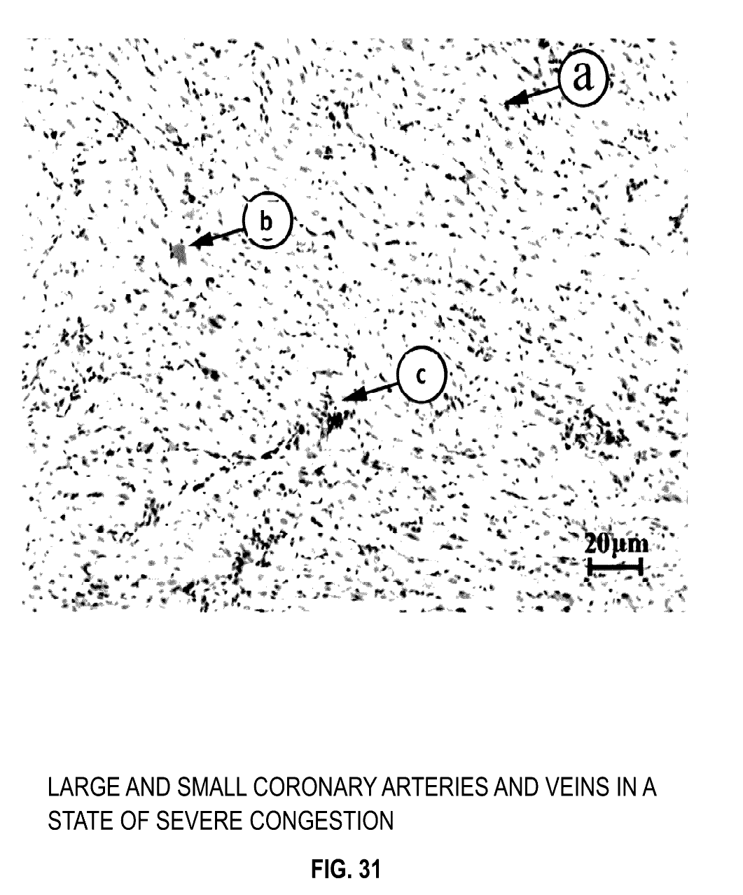

Also seen in the myocardium is clear manifestation of cardiac arrhythmias in the form of numerous muscle fibers in a state of dissociation, fragmentation, and a wave-like deformation (FIG. 31a).

Large and small coronary arteries and veins in a state of severe congestion are presented in sections (FIG. 31b, FIG. 31a). Plasmatic saturation of the arterial wall, sclerosis of the adventitia with a moderately large perivascular field of connective tissue are observed. The endothelium of the blood vessels appears “corrugated” and thickened and peels off in places, and also has protrusions inside the vessel. In the capillaries a sludge of erythrocytes was seen, many of which had gone beyond the vascular bed leading to the development of minor areas of hemorrhaging.

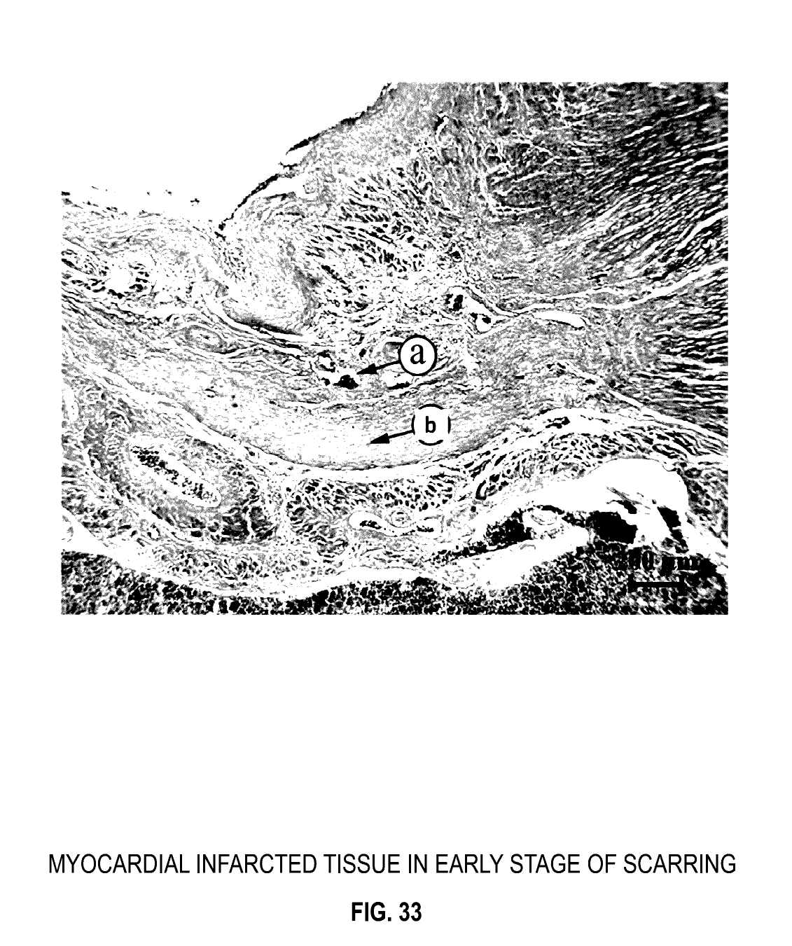

FIG. 33. MI group. Scarring (v×4, approx. ×10): a—in a state of blood supply to the capillaries, and b—connective scar tissue.

When stained by Masson Method, an image of myocardial infarction in the early stage of scarring was observed. Large areas of the myocardium were replaced by maturing unformed loose connective tissue with strong proliferation of fibroblasts (FIG. 33).

SC Group

In the group of rats where experimental myocardial infarction was carried out and treated with stem cells at day 14 histological preparation of the heart wall is represented by three well-distinguishable layers: the inner (endocardium), medium (myocardium) and outer (epicardium). The morphology of the left and right ventricle is different. In the left ventricle, discernible connective scar tissue was observed (FIG. 33b). The scar tissue was wedge-shaped or oblong, at all depths within all layers of the heart wall.

FIG. 33. MI group. Scarring (v×4, approx. ×10): a—in a state of blood supply to the capillaries, and b—connective scar tissue.

When stained by Masson Method, an image of myocardial infarction in the early stage of scarring was observed. Large areas of the myocardium were replaced by maturing unformed loose connective tissue with strong proliferation of fibroblasts (FIG. 33).

SC Group

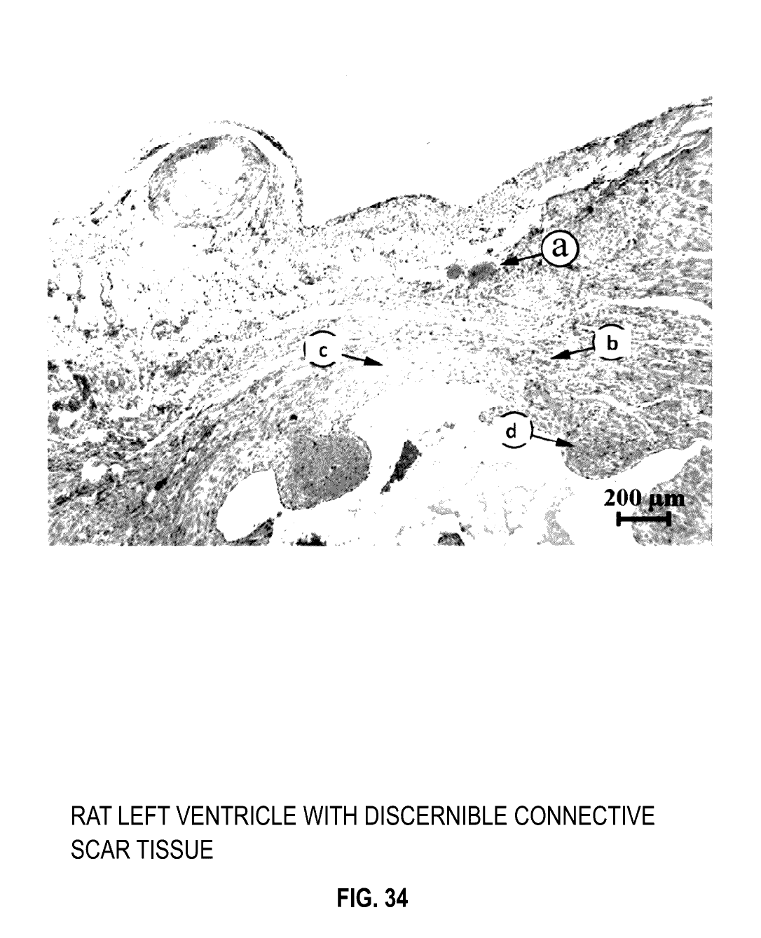

In the group of rats where experimental myocardial infarction was carried out and treated with stem cells at day 14 histological preparation of the heart wall is represented by three well-distinguishable layers: the inner (endocardium), medium (myocardium) and outer (epicardium). The morphology of the left and right ventricle is different. In the left ventricle, discernible connective scar tissue was observed (FIG. 34b). The scar tissue was wedge-shaped or oblong, at all depths within all layers of the heart wall.

FIG. 34. SC group. Scar tissue (v×4, approx. ×10): a—the major blood vessels in a state of increased blood supply, b—capillary blood supply in the state of hypertrophy, B-a connective scar tissue d—cardiomyocytes in a state of hypertrophy. Stained with hematoxylin and eosin.

Scar tissue in the final stage of marutation and is represented by numerous mature fibrocytes and connective tissue cells which have an elongated spindle shape and small hyperchromic rod shaped nucleus.

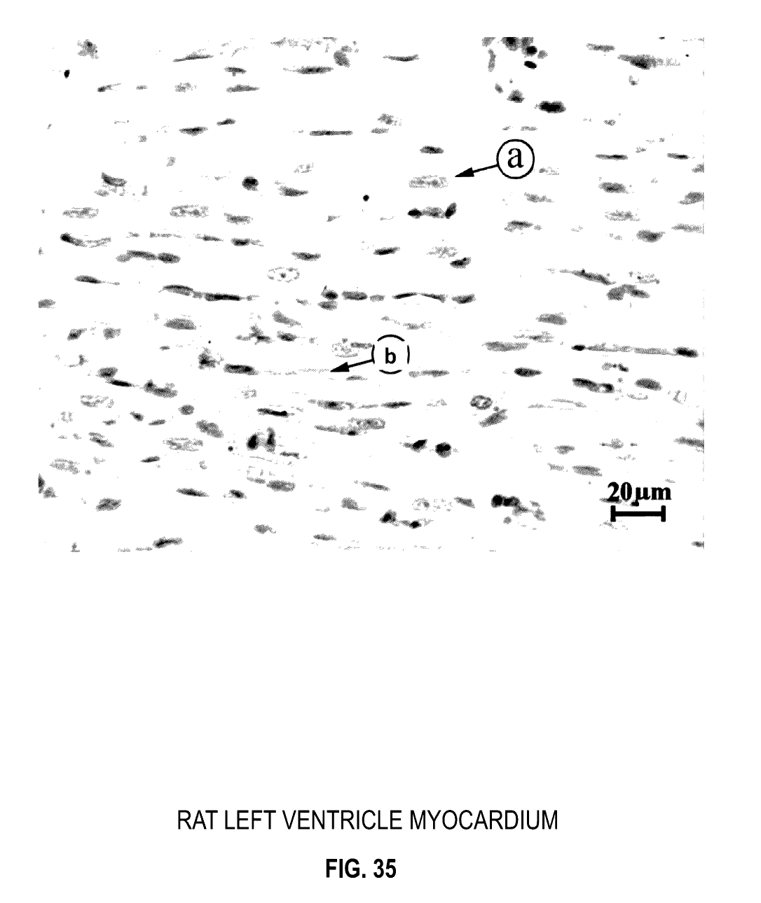

A separate group of cardiomyocytes surrounding connective scar tissue located in the state of hypertrophy (FIG. 34d). They differ in their large size, uneven thickness of the fibers, and polymorphism of the nucleus where some of the cardiomyocytes are round and others are elongated. The majority of the cardiomyocytes of both right and left ventricles are average in size, in proportion to the stained cytoplasm and oval shaped nuclei (FIG. 35a).

FIG. 35. SC group. Myocardium (v×40, ca. ×10): a—a healthy cardiomyocyte b—capillary blood supply in the state of hyperemia. Stained with hematoxylin and eosin.

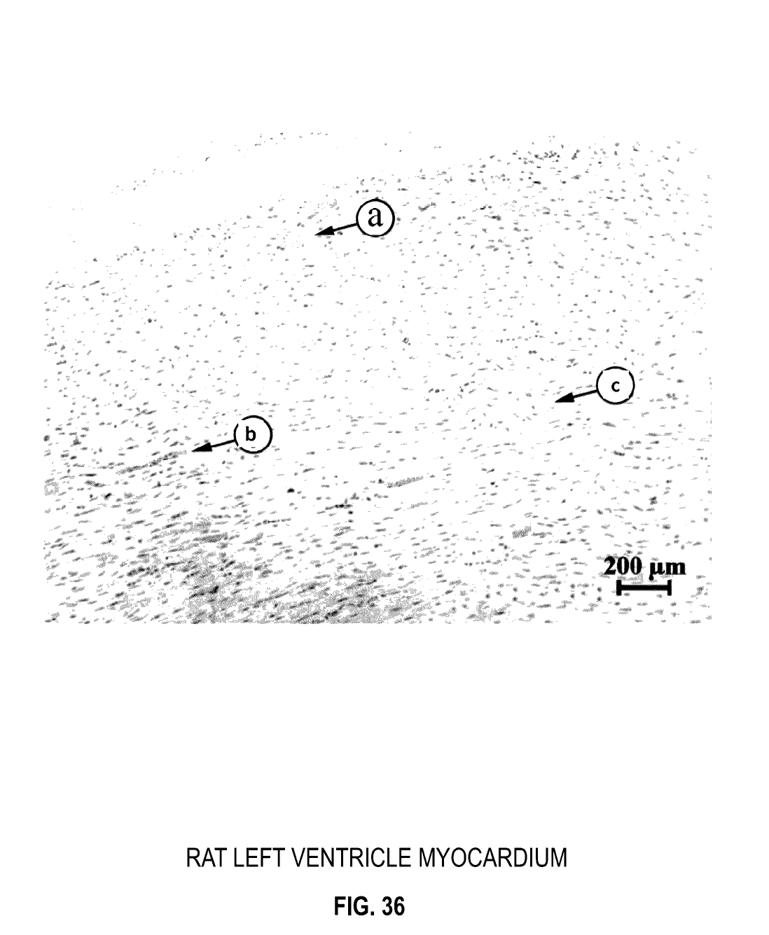

Symptoms of degenerative and necrotic processes in cardiomyocytes are not seen. However, some muscle fibers are in a state of dissociation and fragmentation and a slight wave like deformation is seen which indicates cardiac arrhythmias (FIG. 36b).

FIG. 36. SC Group. Myocardium (v×4, approx. ×10): A—major blood vessels supplied with blood, C—capillaries supplied with blood, and C—a wavy deformation of cardionnyocytes.

Hematoxylin-Eosin Staining

This section contains examples of large and small coronary arteries and veins in a state of rapid decay (FIGS. 34a, b, FIG. 35b, FIG. 36a, b). Plasmorrhagia of the artery walls, sclerosis of the adventitia with moderately large fields of perivascular connective tissue are present. Vascular endothelium appears “corrugated” and thickened, peels off in certain places and has protrusions inside the vessel. In the capillaries sludging of the red blood cells was visible. Around some medium and small-sized vessels small foci of hemorrhaging can be seen.

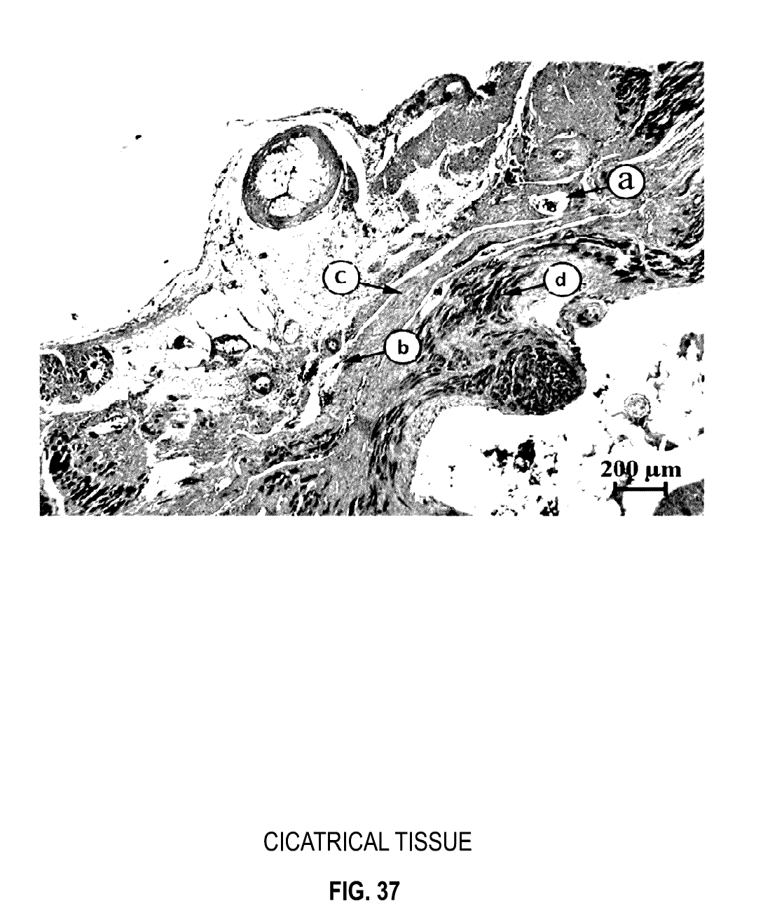

FIG. 37. SC Group. Cicatrical tissue (v×4, approx. ×10): a—the major blood vessels are supplied with blood, b—capillaries are supplied with blood; connective scar tissue d—cardiomyocytes (muscle cells) are in a state of hypertrophy.

When Masson's trichrome stain is used, a pattern of myocardial infarction is observed in the final stages of scarring: large areas of the myocardium have been replaced by maturing unformed loose connective tissue with weak proliferation of fibroblasts and a highly developed intercellular substance (FIG. 37).

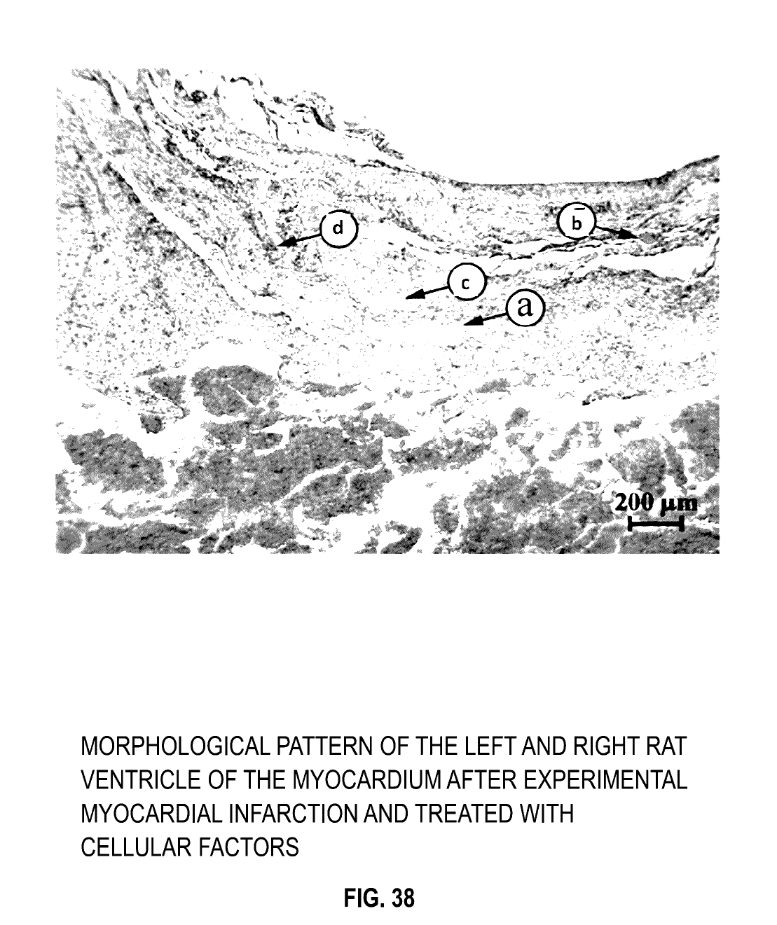

Group CF. On a histological specimen of animals with experimental myocardial infarction treated with cellular factors, on day 14 three well-distinguishable layers are present on the heart wall: the inner (endocardium), medium (myocardium) and outer (epicardium). The morphological pattern of the left and right ventricle of the myocardium are different. In the myocardium on the left ventricular, an area of myocardial infarction can be seen, which has three well-defined components: a segment of cardiomyocyte necrosis, leukocyte infiltration and young unformed loose fibrous connective tissue in a state of maturation (FIG. 38c).

FIG. 38. Group CF. Myocardial (v×4, approx. ×10): a—area of cardiomyocyte necrosis, b—capillary supplied with blood and connective scarring c—connective scarring d—leukocyte infiltration. Hematoxylin-eosin staining.

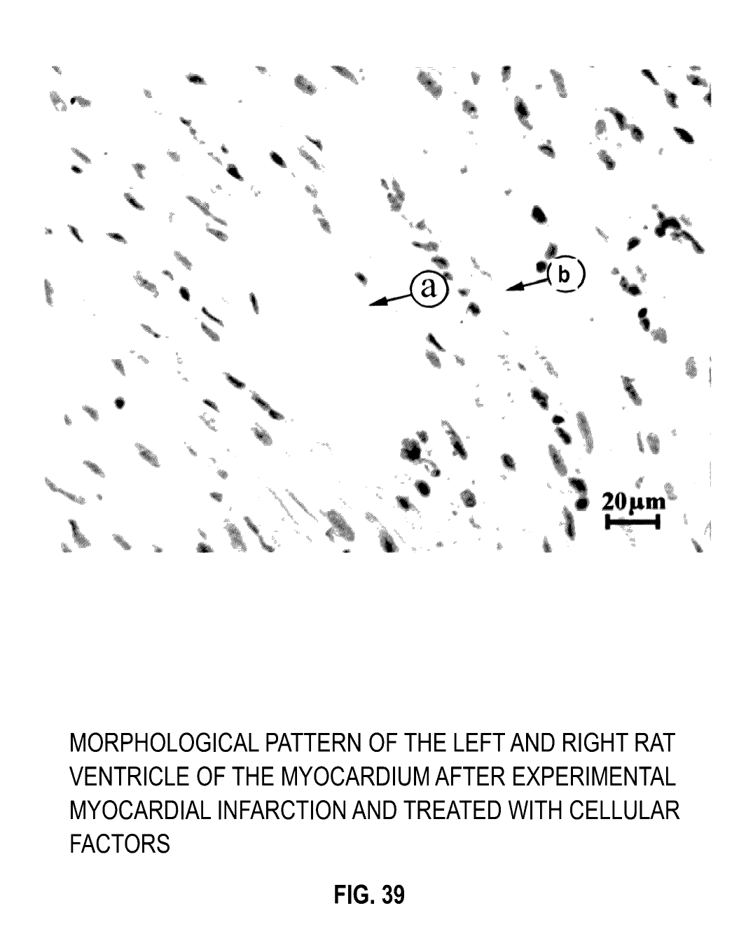

Distinct coagulation necrosis of cardiomyocytes is shown in the form of a single lesion which is less common than several small-sized lesions with distinct boundaries (FIG. 38a). The cardiomyocyte cytoplasm in the necrosis lesion is homogenous and light pink and the nuclei are in a state of karyolysis (FIG. 39a). The cells are fragmented.

FIG. 39. Group CF. Myocardium (v×40, ca. ×10): a —cardiomyocyte with karyolysis b—granular dystrophy cardiomyocytes. Hematoxylin-eosin staining.

Around the necrotic lesion, demarcation inflammation can be seen with infiltration of surrounding necrotic tissue by neutrophils and individual macrophages. Bundles of muscle fibers adjacent to the infarction site in this area are thinned; there is a pronounced swelling of intermuscular stroma with mild leukocyte infiltration (FIG. 38d). The wider area located on the border between the demarcation inflammation and healthy cardiomyocytes contains granulation tissue in a state of maturation.

Granulation tissue consists of fibroblasts having elongated fusiform, and fibroblasts having a multibranched shape that points to other developing repair processes.

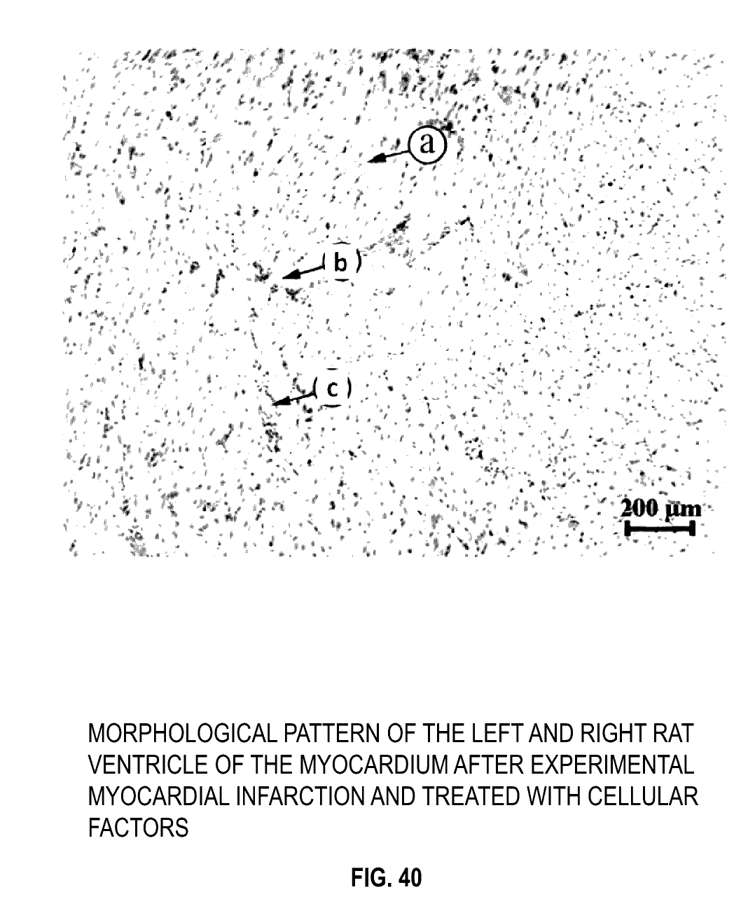

Individual cardiomyocytes surrounding the connective scar formation are in a state of hypertrophy and are distinguishable by their large size, uneven thickness of their fibers and polymorphism of the nucleus, which was part of the same cardiomyocytes. The others have an elongated shape. The majority of the cardiomyocytes of both the right and left ventricles of the heart are average in size and contain a relatively uniformly stained cytoplasm and oval normochromic nuclei. Symptoms of mild degenerative processes in the form of granular cytoplasm of individual cardiomyocytes are present (FIG. 39b and FIG. 40b). However, many of the muscle fibers are in a state of dissociation, fragmentation and undulating deformation indicating an abnormal heart rhythm (FIG. 40a).

FIG. 40. Group CF. Myocardium (v×10, approx. ×10): a—a wavy deformation of cardiomyocytes, b—leukocyte infiltration, c—capillaries supplied with blood. Hematoxylin-eosin staining.

These sections includes large and small coronary arteries and veins in a state of acute plethora (FIG. 38b; FIG. 40c). Plasmatic impregnation of the arteries walls, sclerosis of the adventitia with moderately large fields of perivascular connective tissue are present. Vascular endothelium appears “corrugated” and thickened, peels off in certain places and has protrusions inside the vessel. In the capillaries sludging of the red blood cells was visible, many of which have gone beyond the limit of the vascular bed to the development of small hemorrhagic foci.

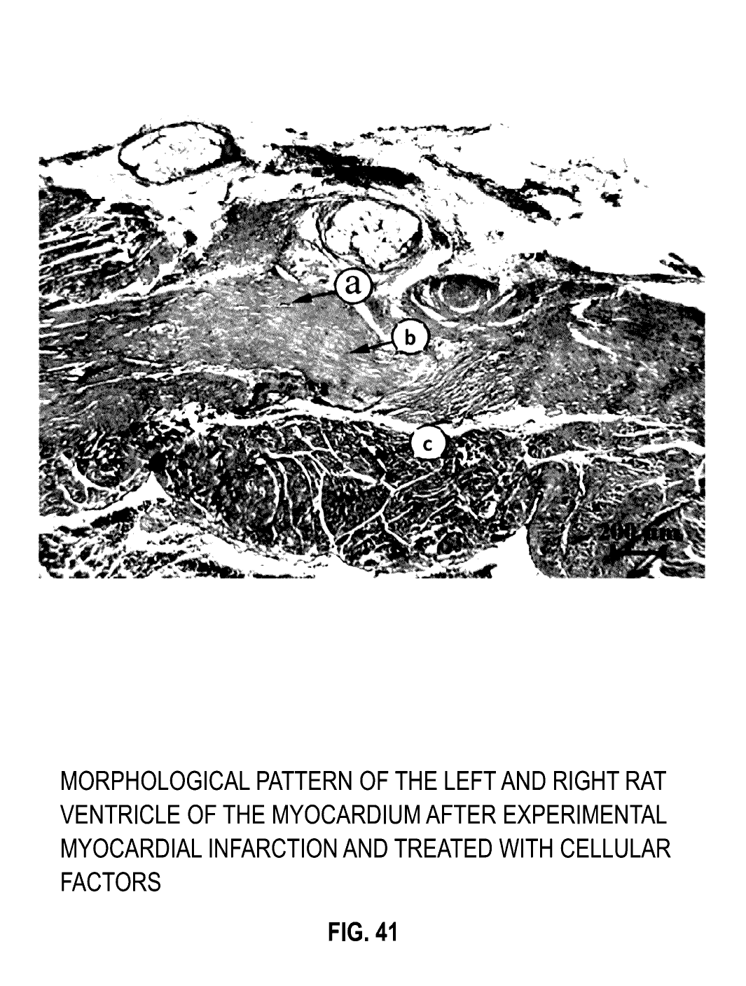

FIG. 41. Group CF. Myocardial (v×4, approx. ×10): A—Capillaries are supplied with blood, B—connective scarring, C—in a state of cardiac hypertrophy.

When Masson's trichrome stain is used, a pattern of myocardial scarring can be seen: large areas of the myocardium have been replaced by maturing unformed loose connective tissue with moderate proliferation of fibroblasts (FIG. 41).

| Symptoms | IM | CS | CF |
|---|---|---|---|
| Patchy capillary- | − − − − − | − − − − − − | − − − − − |
| venous congestion | | | |
| Capillary venous | +++ | ++ | ++ |
| congestion | | | |
| Diffuse | | | |
| Diapedetic | ++ | + | + |
| hemorrhaging | | | |
| Intravascular | +++ | +++ | +++ |
| Uneven coloring of | ++ | − − − − − | + |
| cardiomyocytes | | | |
| Cardiomyocyte | ++ | ++ | ++ |
| hypertrophy | | | |
| Pockets of | + | − − − − − | + |
| fragmentation of | | | |
| muscle fibers infarction | | | |
| The wavy deformation | ++ | + | + |
| of muscle fiber | | | |
| infarction | | | |
| The presence of | ++ | + | + |
| contractures | | | |
| Karyolysis | ++ | − − − − − | + |
| Dissociation of muscle | ++ | + | + |
| fibers | | | |
| Gelatinous fibers | ++ | +++ | +++ |
| Maturation of | ++ | +++ | +++ |
| cicatricial tissue | | | |
| Fibrocyte | ++ | +++ | +++ |
| Fibroblasts | +++ | + | ++ |
| Leukocyte infiltration | ++ | − − − − − | + |

To assess the degree of morphological symptoms, the following conditional criteria were used:

(−)—Symptom is not expressed;

(+)—Symptom is poorly expressed;

(++)—Symptom is moderately expressed;

(+++)—Symptom is strongly expressed.

Thus, the main differences between the groups of animals treated are the degree of scar tissue development, which is almost completely formed in the group of animals SC which received stem cells. None of the animals of this group (n=11) in the myocardium slides showed any signs of necrosis. In the groups CF treated with cellular factors, the scar tissue is in the process of maturation, granulation and resorption of the necrotic masses. Also the reaction is set around the cardiomyocytes which the group of animals treated with the stem cells only manifests signs of arrhythmia, whereas in the groups of animals treated with cellular factors degenerative processes are present.

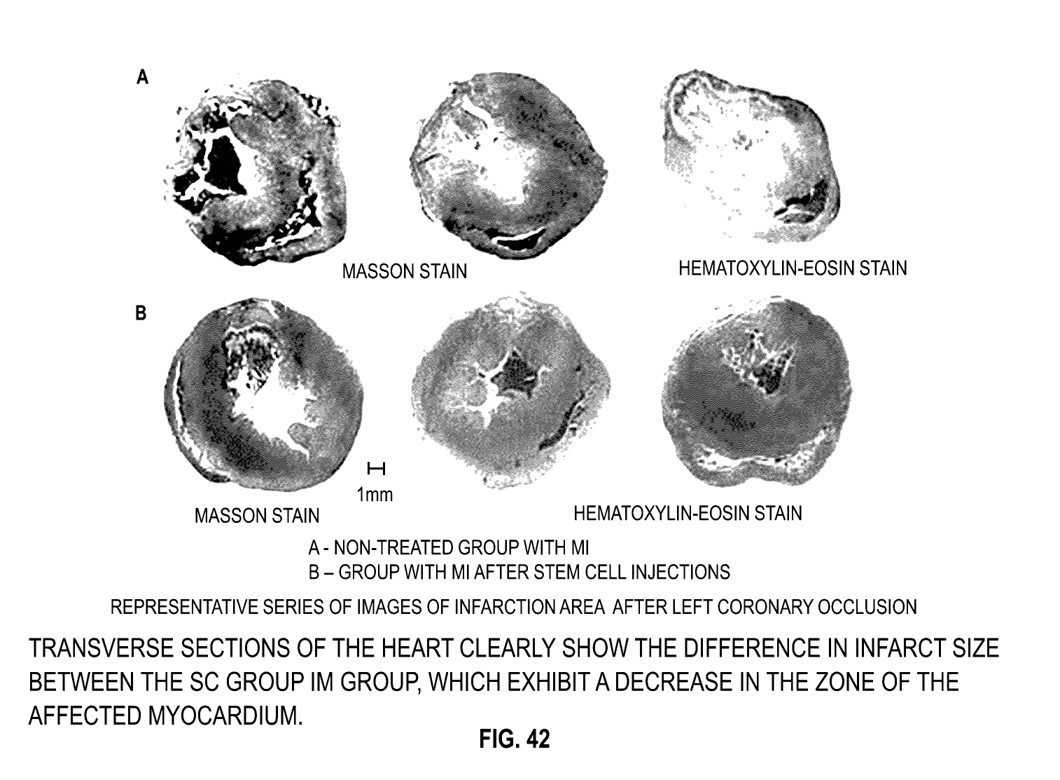

In the macro photographs the hazardous environment was qualitatively assessed, morphometric analysis was performed and the extent of infarction, the level of dilatation of the cavities of the heart and the functioning area of the myocardium and scar tissue were quantitatively measured. Closeups on transverse sections of the heart (FIG. 42) clearly show the difference in infarct size between the SC group IM group, which exhibit a decrease in the zone of the affected myocardium.

FIG. 42 shows transverse sections of the hearts of various animals 14 days after occlusion of the left main coronary artery: A—IM group without treatment, B—Group SC.

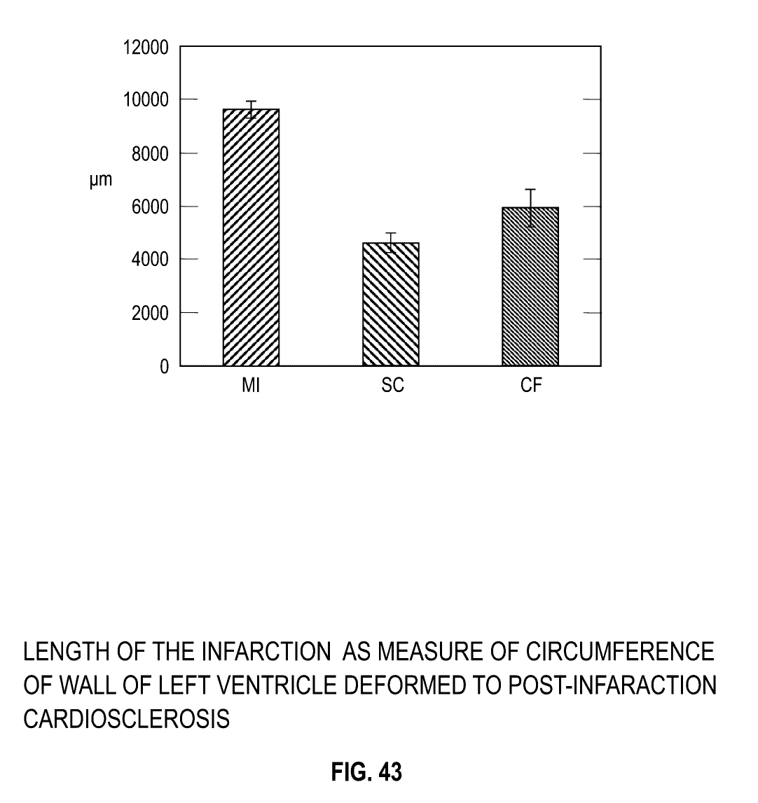

A morphometric analysis confirmed these differences quantitatively. The length of the infarct was assessed by measuring the circumference of the wall of the left ventricle of the heart, which is deformed due to postinfarction cardiosclerosis (FIG. 43). The use of stem cells in the SC group reduces the length of the infarct by 2 times compared to the IM group of animals receiving saline. The histogram also shows a significant decrease of this indicator in groups CF by 38.6%.

FIG. 43. The length of the infarction (heart attack).
Comparison with heart attacks, p<0.001 (Mann-Whitney test).

| Group of rats | |
|---|---|
| MI | 9629.23 ± 331.05 |
| Stem Cells | 4612.90 ± 368.24 |
| Cell Factors | 5917.27 ± 550.85 |

The extent of the infarct was calculated as the ratio of the infarct area in relation to the area of the left ventricle, expressed in percentage (FIG. 28).

And according to this indicator a significant difference in terms of decreasing in all groups with treatment: SC, CF, compared to the MI group by 58.3%, 48.9%, respectively. A similar detailed morphometric study of the parameters that characterize the so-called “expansion” of the heart attack was carried out to evaluate the effect of different periods of reperfusion to preserve the myocardium (Hochman J S, 1987).

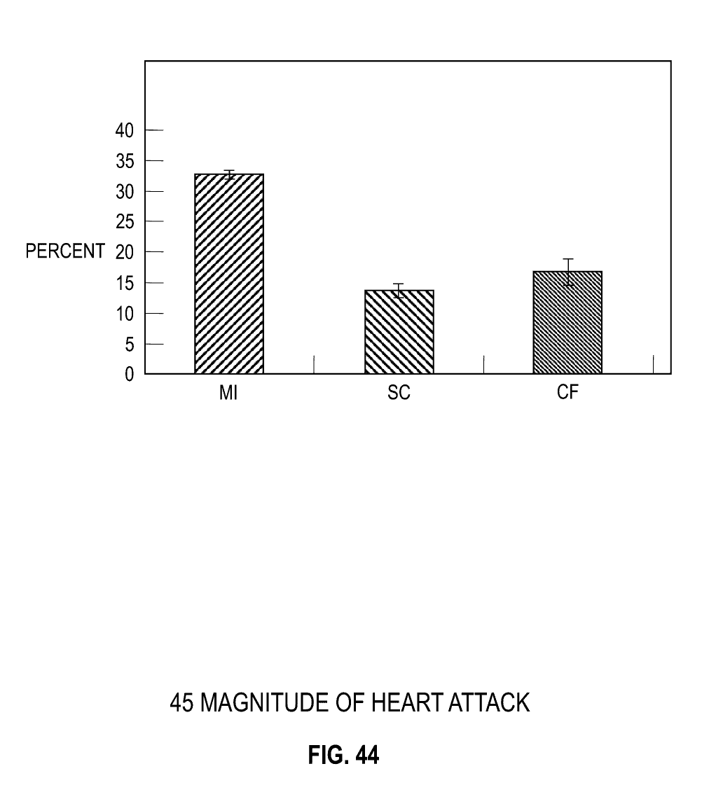

FIG. 44. Magnitude of heart attack. ***

!—Comparison to heart attack, p<0.001

| Groups of rats | % |
|---|---|
| MI | 32.69 ± 0.70 |
| Stem Cells | 13.64 ± 1.14 |
| Cell Factors | 16.71 ± 1.57 |

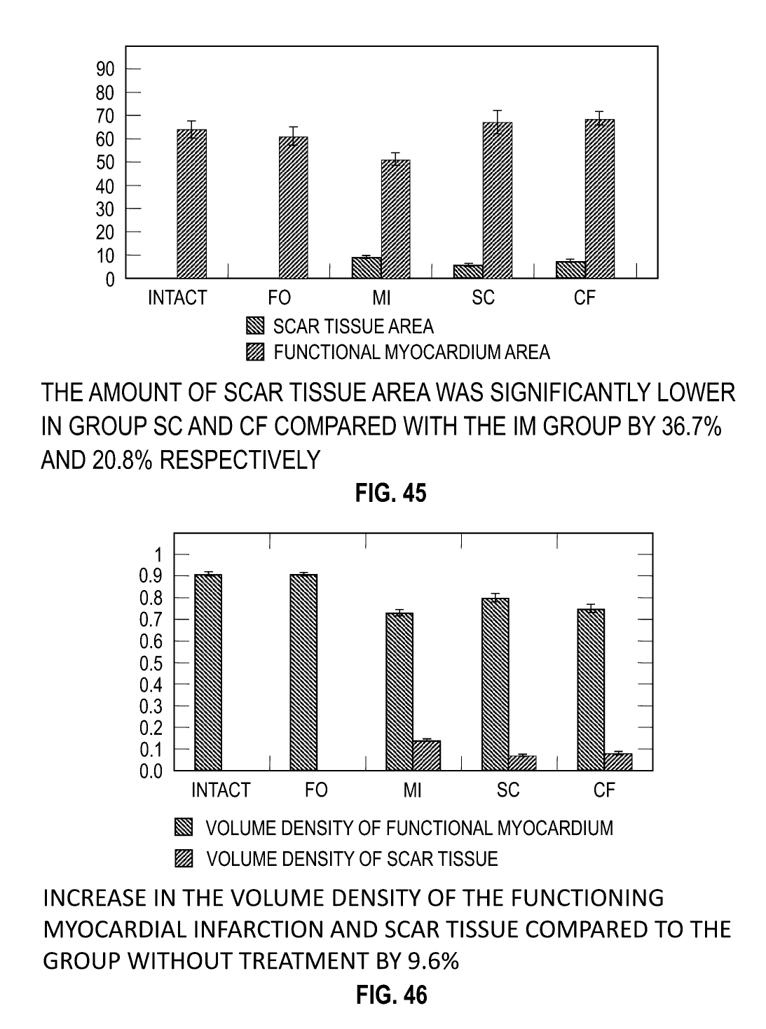

The size of the functioning myocardial area in absolute values was higher in the SC group by 31.2% an in the CF group by 34.1%, compared with the group without the treatment of myocardial infarction, and tended to increase in these groups as compared to the intact control group by 5% and 7.2% respectively. The amount of scar tissue area was significantly lower in Group SC and CF compared with the IM group by 36.7% and 20.8% respectively (FIG. 45).

FIG. 45. Absolute values of the scar tissue and functional myocardium areas.

| | | Area of functioning |
|---|---|---|
| Groups of rats | Area of scar tissue | myocardium |
| Intact | 0 | 63.85 ± 3.59 |
| FO | 0 | 61.03 ± 3.96 |
| MI | 9.22 ± 0.37 | 51.07 ± 2.68 |
| Stem Cells | 5.84 ± 0.31 | 66.99 ± 4.99 |
| Cell Factors | 7.30 ± 0.56 | 68.46 ± 2.88 |

A volume density indicator of the scar tissue reveals a significant reduction of this parameter in all the experimental groups of animals (SC—50%, and CF—42.9%), compared to the IM group. Similar data on the reduction of myocardial infarction was obtained in the treatment of experimental myocardial infarction with recombinant human granulocyte colony-stimulating factor (E D Goldberg, 2006).

Furthermore, in the group treated with stem cells, there was an increase in the volume density of the functioning myocardial infarction compared to the group without treatment by 9.6% (FIG. 46). This indicates more severe postinfarction cardiac hypertrophy in the SC group and is possibly an additional consequence of cell therapy.

FIG. 46. The volume density of functioning myocardium and scar tissue. *** comparison with a heart attack, p<0.001 (Mann-Whitney test).

| | Volume density of functioning | Volume |
|---|---|---|
| Groups of rats | myocardium | density of scar tissue |
| Intact | 0.91 ± 0.007 | 0 |
| FO | 0.91 ± 0.007 | 0 |
| MI | 0.73 ± 0.015 | 0.14 ± 0.005 |
| Stem Cells | 0.80 ± 0.020 | 0.07 ± 0.005 |
| Cell Factors | 0.75 ± 0.019 | 0.08 ± 0.007 |

Furthermore, we found that the volume density of the cavities in the ventricles in groups SC and CF tended to increase as compared to the control group IM with experimental myocardial infarction. The volume density of the left ventricular cavity in groups SC and CF increased by 27.2% and 18.5%, and the right ventricle—25% and 22.7%, respectively.

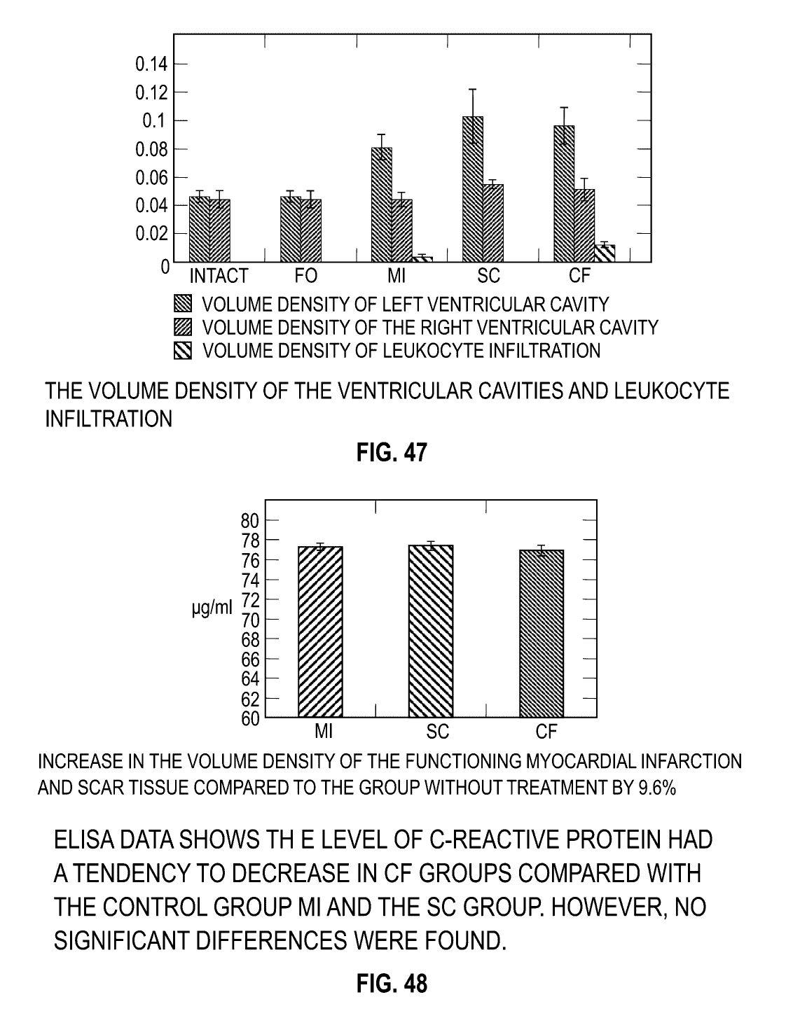

FIG. 47. The volume density of the ventricular cavities and leukocyte infiltration. ***

| | Volume density | Volume density | Volume density |
|---|---|---|---|
| | of left | of the right | of leukocyte |
| Groups of rats | ventricular cavity | ventricular cavity | infiltration |
| Intact | 0.046 ± 0.004 | 0.044 ± 0.006 | 0 |
| FO | 0.046 ± 0.004 | 0.044 ± 0.006 | 0 |
| MI | 0.081 ± 0.009 | 0.044 ± 0.005 | 0.004 ± 0.001 |
| Stem Cells | 0.103 ± 0.019 | 0.055 ± 0.003 | 0 |
| Cell Factors | 0.096 ± 0.013 | 0.051 ± 0.008 | 0.012 ± 0.002 |

Hypertrophy and dilatation of the heart cavities occur in response to a dysfunction of the left ventricle, which arose as a result of irreversible myocardial damage after occlusion of the descending branch of the left main coronary artery. Compensatory dilatation aims to restore and maintain the stroke volume of the pumping function of the heart by decreasing the mass of the diminishing infarction. Thus, cardiomyocyte hypertrophy is aimed at strengthening the ventricular wall which experiences a significant increase in stress due to dilation. However, compensatory dilation when there is a significant amount of damage and inadequate hypertrophy can lead to greater dilation. In this case, these compensatory processes can lead to a (progression) exacerbation of dysfunction.

None of the animal groups treated with stem cells (n=11) in the microscopic sections of the myocardium showed signs of necrosis. Therefore, we did not present data on the differences in the volume density of necrotic tissue. The volume density of leukocyte infiltration into the SC group is so insignificant that it is not visible on the chart. However, the volume density of leukocyte infiltration into the CF group increased, compared with the SC group (FIG. 47).

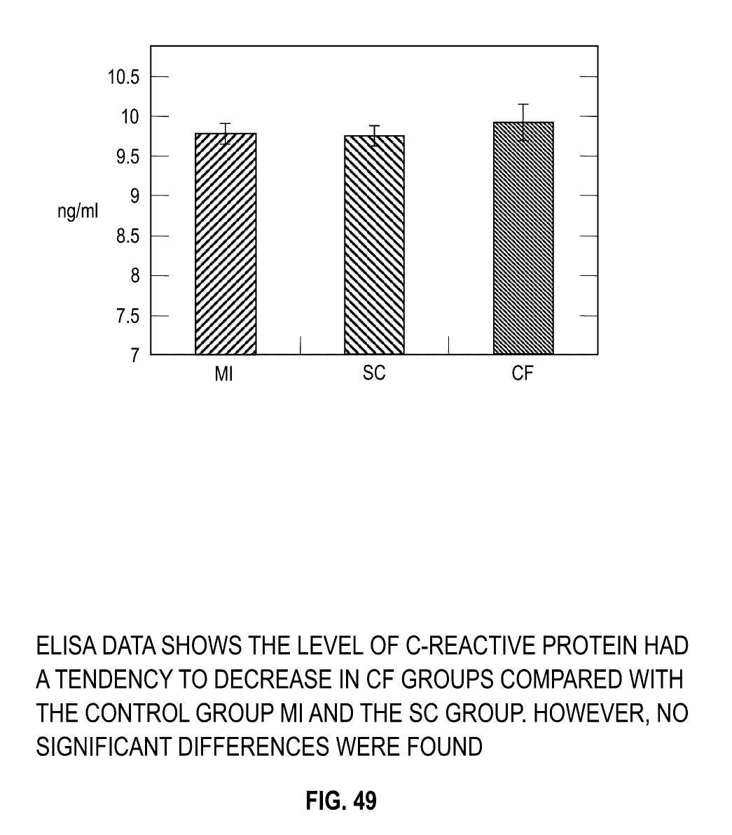

ELISA data is shown in FIG. 48 and FIG. 49. The level of C-reactive protein had a tendency to decrease in CF groups compared with the control group MI and the SC group. However, no significant differences were found.

FIG. 48. C-reactive protein in the serum of different groups of animals with experimental myocardial infarction (14 days after coronary artery occlusion). ***

| Group of rats | μg/ml |
|---|---|
| MI | 77.28 ± 0.39 |
| Stem Cells | 77.37 ± 0.44 |
| Cell Factors | 76.87 ± 0.40 |

Levels of brain natriuretic peptide in the serum also did not differ between the treatment groups of animals.

FIG. 49. Levels of natriuretic peptide in serum of animals of different experimental groups IM (14 days after coronary artery occlusion).

| Groups of rats | ng/ml |
|---|---|
| MI | 9.77 ± 0.13 |
| Stem Cells | 9.75 ± 0.13 |
| Cell Factors | 9.92 ± 0.23 |

Classical protein in an acute phase of inflammation (CRP) is an extremely sensitive marker of disease in clinical practice for monitoring and differential diagnosis. Inflammation as a factor in the synthesis of CRP plays a key role in the pathogenesis of cardiovascular diseases. Raising the level of CRP in serum indicates acute myocardial infarction. (Yeh E T., 2003).

Brain natriuretic peptide (BNP) is considered a marker of the functional condition of the contractile capacity of the heart muscle. An increase in BNP levels indicates heart failure; left ventricular hypertrophy: inflammation of the heart tissue—myocarditis, acute coronary syndrome, acute myocardial infarction (BNP release due to tissue necrosis).

However, depending on the duration of the disease, levels of both CRP and BNP significantly change.

According to different data, the level of C-reactive protein is elevated in acute myocardial infarction (appears on the 2nd day of the disease and by the end of the 2nd week/early part of the 3rd week disappears from the serum). The maximum concentration of CRP is observed on the first day of acute myocardial infarction and decreases to almost normal by day 10 (De Kam P J, 2002).

Some studies have suggested that BNP has less sensitivity to predict left ventricular dysfunction compared to heart failure diagnosis, particularly in the case of mild dysfunction (Seino Y., 2004; Hunt P J., 1997; Nishikimi, T., 2006).

### CONCLUSIONS

In the SC group of animals which received stem cells, scar tissue is more mature. There is no foci of necrosis. There is no degeneration of cardiomyocytes.

In the SC group of animals which received stem cells, the myocardial infarction zone decreased. There was a 2-fold decrease in the length of a heart attack in the SC group; it fell in the group of CF by 38.6%. The vastness of a heart attack decreased in groups SC, CF by 58.3% and 48.9%, respectively.

The volume density of the scar tissue of the animals in all three experimental groups decreased compared to the control group: in the SC by 50%, CF by 42.9%. In other words, the scarring itself decreased.

In the group treated with stem cells, there is a slight (9.6%) increase in the volume density of the functioning myocardium compared to the IM group, indicating more severe myocardial hypertrophy. Furthermore, there was a trend towards an increase in ventricular dilatation in groups SC and CF.

ELISA revealed no significant differences between the groups in the level of C-reactive protein and brain natriuretic peptide peptide in the serum 14 days after occlusion LCA.

The examples reported herein support that MSCs delivered intravenously exerted biologically relevant therapeutic effects on the limbs of subjects with peripheral arterial disease and hindlimb ischemia. These findings demonstrated that there was an initial localization of MSCs in lung, liver and heart, followed by gradual release of the ivMSCs that are acutely entrapped in the lungs and other tissues, which lead to redistribution so that an increased number of human MSCs localize and engraft into ischemic tissue over the several days following intravenous injection, which is efficacious to recovery of ischemic hindlimb muscle. The examples taken together indicate that a composition of itMSCs comprising or not comprising stem cell factors, delivered intravenously, or delivered by combining administration intravenously and intramuscularly exerted biologically relevant therapeutic effects on the limbs of subjects with peripheral arterial disease and hindlimb ischemia.

### LITERATURE

Avtandilov G G Medical morphometry G. G. Avtandilov.—Moscow: Nauka, 1990.-340 with Avtandilov G G Fundamentals of quantitative pathology G. G. Avtandilov.—Moscow, Medicine, 2002.-238.
Goldberg E D, Dygai A M, Vladimir Zhdanov, Stavrova L A, T I Fomin, Plotnikov, M B, Aliev C H, Masycheva V I Sotnikov H. B. The method of experimental therapy of myocardial infarction. Russian Patent R U (11) 2288738 (13) C2.
Dubovik, B V, A A Zhdanov, Romanovsky D. Comparison of cardioprotective efficacy of arginine hydrochloride, arginine and succinate in ischemia-reperfusion injury in rats//Journal of Medicine.—2005.—N 4.—S. 51-53.
The European Convention for the Protection of Vertebrate Animals used for experimental and other scientific purposes. Questions and reconstructive plastic surgery, 2003.-N 4.-P. 34-36.
Lilly, R. histopathological technique and practical histochemistry. Springer-Verlag, 1969.—645. Merkulov, G A Course pathohistological techniques. AL: Medicine, 1969. 340.
Euthanasia of experimental animals (Guidelines for the removal of animals from the experiment). Moscow: USSR Ministry of Health, 1985.
Kam P. J., Nicolosi G. L., Voors A. A. et al. Prediction of 6 months left ventricular dilatation after myocardial infarction in relation to cardiac morbidity and mortality: Application of a new dilatation model to GISSI-3 data//Eur. Heart J., 2002, Vol. 23, P. 536-542.
Hochman J S, Choo H. Limitation of myocardial infarct expansion by reperfusion independent of myocardial salvage. Circulation. 1987 January; 75(1):299-306.
Hunt P J., Richards A M., Nicholls M G., Yandle T G., Doughty R N., Espiner E A. Immunoreactive amino-terminal pro-brain natriuretic peptide (NT-PROBNP): a new marker of cardiac impairment. Clin. Endocrinol. (Oxf). 1997; 47(3): 287-96.
Nishikimi T, Maeda N, Matsuoka H. The role of natriuretic peptides in cardioprotection-Toshio Nishikimi, Nobuyo Maeda, Hiroaki Matsuoka—Cardiovasc Res., 2006, 69(2): 318-328.
Seino Y., Ogawa A., Yamashita T., Fukushima M., Ogata K., Fukumoto H., Takano T. Application of NT-proBNP and BNP measurements in cardiac care: a more discerning marker for the detection and evaluation of heart failure. Eur. J. Heart Fail. 2004; 6: 295-300.
Srikanth G V N, Prakash P, Triathy N K, Dikshit M, Nityanand S. Establishment of a rat model myocardial infarction with a high survival rate: A suitable model for evaluation of efficacy of stem cell theapy. JSRM, 2009, V.5, 1, 30-36.
Yeh E T, Willerson J T. Coming of age of C-reactive protein: using inflammation markers in cardiology. Circulation. 2003; 107: 370-372.

What is claimed is:

1) A method for treating a peripheral arterial disease (PAD) condition in a patient comprising:
a) providing a stem cell composition;
b) administering the stem cell composition to the patient;
c) wherein administering the stem cell composition treats the PAD condition.

2) The method of claim 1, wherein the stem cell composition is autologous, allogeneic, or a combination thereof with respect to the patient.

3) The method of claim 2 wherein the stem cell composition comprises mesenchymal stem cells.

4) The method of claim 3 wherein said stem cell composition comprises more than one line of stem cells.

5) The method of claim 2, wherein the mesenchymal stem cells are derived from cord blood, bone marrow, peripheral blood, adipose tissue, or embryonic tissue or a combination thereof.

6) The method of claim 5 wherein said mesenchymal stem cells comprise iPS cells.

7) The method of claim 3 wherein the mesenchymal stem cells are derived from bone marrow.

8) The method of claim 3 wherein said mesenchymal stem cells are ischemic tolerant mesenchymal stem cells.

9) The method of claim 1 wherein said stem cell composition comprises stem cell factors.

10) The method of claim 1 wherein the PAD condition is limb ischemia.

11) The method of claim 1, wherein the stem cell composition is administered intravenously.

12) The method of claim 1 wherein the stem cell composition is administered intravenously and intramuscularly.

13) The method of claim 1 wherein the stem cell composition is administered intramuscularly.

14) A method for treating a peripheral arterial disease (PAD) condition in a patient comprising:
a) providing a stem cell factor composition;
b) administering the stem cell factor composition to the patient;
c) wherein administering the stem cell factor composition treats the PAD condition.

15) The method of claim 1, wherein the stem cell factor composition is derived from cells which are autologous, allogeneic, or a combination thereof with respect to the patient.

16) The method of claim 15 wherein the stem cell factor composition is derived from mesenchymal stem cells.

17) The method of claim 15 wherein said stem cell factor composition derives from more than one line of stem cells.

18) The method of claim 15, wherein the stem cell factors are derived from mesenchymal stem cells which are derived from cord blood, bone marrow, peripheral blood, adipose tissue, or embryonic tissue or a combination thereof.

19) The method of claim 14 wherein said stem cell factors are derived from iPS cells.

20) The method of claim 16 wherein the mesenchymal stem cells are derived from bone marrow.

21) The method of claim 3 wherein said mesenchymal stem cells are ischemic tolerant mesenchymal stem cells.

22) The method of claim 14 wherein the PAD condition is limb ischemia.

23) The method of claim 14, wherein the stem cell factor composition is administered intravenously.

24) The method of claim 14 wherein the stem cell factor composition is administered intravenously and intramuscularly.

25) The method of claim 14 wherein the stem cell factor composition is administered intramuscularly.
